(19) United States
(12) Patent Application Publication
(10) Pub. No.: US 2025/0318739 A1
(43) Pub. Date: Oct. 16, 2025

(54) IMPLANTABLE STRETCH SENSOR FOR BLOOD PRESSURE/FLOW MONITORING

(71) Applicant: Case Western Reserve University, Cleveland, OH (US)

(72) Inventors: Steve Majerus, University Hts., OH (US); Jeremiah Ukwela, Cleveland, OH (US)

(21) Appl. No.: 19/175,521

(22) Filed: Apr. 10, 2025

Related U.S. Application Data
(60) Provisional application No. 63/632,141, filed on Apr. 10, 2024.

(51) Int. Cl. A61B 5/021; A61B 5/00; A61B 5/0215; A61B 5/026; A61B 5/107

(52) U.S. Cl. CPC A61B 5/02141; A61B 5/0215; A61B 5/026; A61B 5/107; A61B 5/6876; A61B 5/742; A61B 2560/0462; A61B 2562/0247; A61B 2562/0261; A61B 2562/164; A61B 2562/227

(57) ABSTRACT

An improved, implanted stress sensor can continuously monitor blood pressure/flow, and/or other cardiovascular variability parameters, in ambulatory and non-ambulatory patients. The sensor includes a flexible substrate configured to wrap around at least a portion of an artery. The flexible substrate includes a sensing portion in an intermediate portion of the flexible substrate and a non-sensing portion split between opposite ends of the flexible substrate. The sensing portion includes a piezoresistive material configured to detect changes in a diameter of the artery. The non-sensing portion includes at least one opening positioned on each of the opposite ends of the flexible substrate and a stiffening mesh around each of the at least one openings. One or more closure mechanisms connect the opposite ends of the flexible substrate via the openings. The sensor can include at least one lead to connect the sensing portion and a controller.

### CROSS-REFERENCE TO RELATED APPLICATIONS

This application claims the benefit of U.S. Provisional Application No. 63/632,141, filed 10 Apr. 2024, entitled “FINITE ELEMENT INFORMED OPTIMIZATION OF AN IMPLANTABLE FLEXIBLE PRESSURE SENSOR”, which is incorporated herein by reference in its entirety.

### TECHNICAL FIELD

The present disclosure relates generally to sensors for healthcare applications and more specifically, to an implantable stretch sensor for continuous blood pressure/flow monitoring.

### BACKGROUND

Cardiovascular diseases (e.g., hypertension, atherosclerosis, heart failure, etc.) are a leading global health challenge, consistently ranking as a top cause of mortality and morbidity. Accurate and real-time monitoring of blood pressure/flow is crucial for the management and/or treatment of cardiovascular diseases and other conditions that rely on normal blood flow. Blood pressure serves as a key indicator of cardiovascular function, providing critical insights into an individual's overall health and predicting potential risks associated with cardiovascular diseases. The gold standard in blood pressure measurement is an invasive technique, intra-arterial catheterization, but this invasive technique cannot be used to perform continuous monitoring in ambulatory patients. Additionally, invasive methods (not limited to intra-arterial catheterization) have a myriad of challenges and limitations including but not limited to risks of infection, hemorrhage, thrombosis, or the like as well as patient discomfort and stress that can cause inaccurate results. Non-invasive approaches can continuously estimate blood pressure in real time, ambulating patients using optical methods (such as photoplethysmography (PPG)) or sound based methods (such as ultrasonic sensors). However, non-invasive sensors rely on patient compliance to keep the devices in working order, and accuracy and reliability of measurements can be impaired by motion artifacts, ambient light interference, skin color, improper calibration, or the like.

### SUMMARY

The present disclosure describes an improved implantable stretch sensor for measuring cardiovascular parameters, such as blood pressure and/or blood flow. Compared to similar sensors, the implantable stretch sensor (referred to as “sensor”) described herein can provide more accurate and reliable results for chronic implantation and can better resist wear and tear from continuous use and implantation.

In one aspect, the present disclosure includes an implantable sensor for measuring at least blood pressure in an artery. The sensor includes a flexible substrate configured to wrap around at least a portion of the artery. The flexible substrate includes a sensing portion in an intermediate portion of the flexible substrate and a non-sensing portion split between opposite ends of the flexible substrate. The sensing portion includes a piezoresistive material that includes conductive particles suspended in a polymer and configured to detect changes in a diameter of the artery. The non-sensing portion includes at least one opening positioned longitudinally on each of the opposite ends of the flexible substrate and a stiffening mesh around each of the at least one opening on each of the opposite ends of the flexible substrate. One or more closure mechanisms connect the opposite ends of the flexible substrate via the at least one opening on each of the opposite ends of the flexible substrate. The sensor further includes at least one lead configured to interface with the sensing portion to transmit the detected changes in the diameter of the artery to a controller.

In another aspect, the present disclosure includes a system comprising a sensor and a controller for continuous monitoring of at least blood pressure. The sensor includes a flexible substrate configured to wrap around at least a portion of the artery. The flexible substrate includes a sensing portion in an intermediate portion of the flexible substrate and a non-sensing portion split between opposite ends of the flexible substrate. The sensing portion includes a piezoresistive material including conductive particles suspended in a polymer and configured to detect changes in a diameter of the artery. The non-sensing portion includes at least one opening positioned longitudinally on each of the opposite ends of the flexible substrate and a stiffening mesh around each of the at least one opening on each of the opposite ends of the flexible substrate. One or more closure mechanisms connect the opposite ends of the flexible substrate via the at least one opening on each of the opposite ends of the flexible substrate. The controller is in communication with the sensor and includes at least a processor to execute instructions to determine measurements related to arterial pressure (e.g., blood pressure/flow) based on recordings from the sensor and output the measurements related to the arterial pressure.

In a further aspect, the present disclosure includes methods for implanting, fabricating, and using the sensor and/or the system for continuous monitoring of at least blood pressure.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a block diagram of a system for measuring blood pressure and/or flow using an implantable stress sensor;

FIG. 2 is an illustration of an inner face of a sensor of the system of FIG. 1;

FIGS. 3 and 4 are illustrations showing zoomed in views of cut-line portions of the sensor of FIG. 2.

FIG. 5 is an illustration of an example sensor of the system of FIG. 1 in a wrapped configuration;

FIG. 6 is an illustration of how the sensor of FIG. 5 is effected by blood pressure and/or flow in an artery;

FIGS. 7 and 8 are illustrations of example sensors that can be used in the system of FIG. 1 in two configurations with closure mechanisms;

FIGS. 9-11 are process flow diagrams illustrating methods related to the stress sensor; and

FIGS. 12-40 show pictures, graphs, and models used in the experiments to create the stress sensor.

### DETAILED DESCRIPTION

### I. Definitions

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present.

As used herein, the term “stretch sensor” refers to a sensor that can measure deformation or stretching forces in terms of electrical conductivity. Such a sensor can include a strain gauge that exhibits changes in electrical resistance when subject to one or more mechanical forces that deform the strain gauge in at least one direction (e.g., stretches the strain gauge). In some instances, the strain gauge can include a piezoresistive material so that the strain gauge has piezoresistive qualities. In some instances, the stretch sensor can be built on a substrate.

As used herein, the term “stress” refers to a measure of an intensity of internal forces that arise within a material when the material is subjected to external loads or forces. Stress is traditionally measured as force per unit area. Examples of types of stress include tensile stress, compressive stress, shear stress, torsional stress, and the like.

As used herein, the term “strain” refers to a measure of the deformation or change in shape of a material from a reference position when subjected to an external force. Examples of types of strain can include deformation of a shape and/or a size of an object, which can include stretching, compression, shearing, or the like.

As used herein, the term “stretching” refers to an ability of a material that is soft and/or elastic being able to lengthen or widen without tearing and/or the act of lengthening and/or widening without tearing.

As used herein, the term “piezoresistive” refers to a material property where an electrical resistance of the material changes when subjected to mechanical stress or strain (known as the piezoresistive effect). Example of piezoresistive materials include, but are not limited to, semiconductors, metal alloys, conductive polymers, carbon-based materials, and the like. piezoresistive materials can be any size and/or shape.

As used herein, the term “piezoresistive elastomer composite” refers to piezoresistive materials (e.g., conductive particles such as carbon black particles and/or carbon nanotubes) being suspended in a polymer material to form a composite.

As used herein, the term “substrate” refers to a foundational material or structure that provides structural support and/or electrical connectivity to one or more components built thereon. Examples of the components can include electronic devices, circuits, other elements (such as one or more piezoresistive strain gauges, one or more leads, etc.), or the like. In some instances, the substrate can be formed, at least in part, from a flexible material so that at least a portion of the substrate can wrap at least partially around an artery.

As used herein, the term “artery” refers to a blood vessel that distributes oxygen rich blood away from a patient's heart to tissues and organs in the patient's body. Examples of arteries include, but are not limited to, the aorta, the pulmonary artery, the common carotid artery, the brachiocephalic artery, the subclavian artery, the femoral artery, the radial artery, and the renal artery.

As used herein, the term “blood pressure” refers to the force of blood pushing against the walls of an artery. Blood pressure that is outside normal ranges (high or low) can be an indicator of many diseases, including but not limited to, cardiovascular disease, stroke, heart attack, heart failure, aneurysms, or the like. Blood pressure may be used alone, or in combination with other physiological measurements to estimate other cardiovascular variability parameters (e.g., heart rate variability (HRV). SDNN (standard deviation of NN intervale), RMSSD (root mean square of successive differences between NN intervals), low frequency components, high frequency components, etc.).

As used herein, the term “blood flow” refers to the movement of blood through an artery or vessel. Proper blood flow is vital for metabolism and overall health, ensuring that oxygen and nutrients are delivered to cells and that waste products are removed from cells. Measuring blood flow can help diagnose and monitor conditions. For example, the amount of blood pumped with each heartbeat can be a sign of how large a vessel's opening is and can indicate abnormal blockages. Generally, higher blood pressure can lead to increased blood flow.

As used herein, the term “closure mechanism” refers to a mechanism that can fasten two sides of a substrate of a stretch sensor together to at least partially encircle an artery. Examples of the closure mechanism can include rivets, such as snap rivets, sutures, adhesives, such as UV curing adhesives, or the like that can be biocompatible.

As used herein, the term “snap-rivet” refers to a fastener that has a male component and a female component that snap together to hold one or more materials together. The male component can include one or more portions that expand and cause the catching. The female component can include a receiving element (e.g., a hole) that the male component can be pushed through. As an example, the male component of the snap rivet can be pushed through a hole in the female component and catches on a portion of the hole to secure the two components together.

As used herein, the terms “patient” and “subject” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

Accurate and real-time monitoring of blood pressure/flow is crucial for the management of cardiovascular diseases and other conditions that rely on normal blood flow. Such accurate and real time monitoring can be achieved with an implantable sensor. One example of such an implantable sensor is a stretch sensor, referred to as a flexible pulsation sensor (FPS), which can be implanted and wrapped at least partially around an artery (e.g., the carotid artery) to measure blood pressure/flow. The FPS has the capability to bypass external interferences, such as motion artifacts or environmental conditions, which can affect the reliability of non-invasive measurements. The FPS is particularly advantageous in chronic conditions where continuous surveillance is critical. However, previous versions of the FPS have suffered from challenges arising from mechanical failures and signal degradation during chronic implantation (e.g., 30 day implantation or longer, 60 day implantation or longer, 120 day implantation or longer, or the like).

The mechanical failures can be due to mechanical factors, such as the sensor not staying closed, plastic deformation (or permanent distortion) of parts of the sensor, or the like; biological factors, such as fibrotic growths affecting the sensor's readings; electrical factors such as lead connections decaying or breaking; and/or the like. The stretch sensor described herein is an improvement over previous versions of the FPS sensor, targeting the mechanical failures due to mechanical, electrical, and biocompatibility deficiencies of the previous version of the FPS sensor to improve chronic, continuous monitoring of at least blood pressure and/or flow. For instance, improvements in the substrate material to a material with a reduced elastic modulus allow for increased deformation and toughness to increase mechanical longevity in-vivo and increased sensitivity to arterial wall deformation, improvements in the closure mechanism and surrounding material increase tear strength to keep the sensor closed, and changes in the sensing material (e.g., the piezoresistive material) improves elasticity and sensitivity to strain.

### III. Systems

Accurate, continuous monitoring of blood pressure/flow in real time is crucial for the management of cardiovascular diseases and other conditions affected by abnormal blood pressure/flow. Patients and their designated medical professionals need to be aware of both spikes and trends in blood pressure and blood flow to treat and/or manage cardiovascular diseases. Described herein is a flexible implantable sensor (referred to as the stretch sensor) that can conform to the contours of the body and accommodate internal deformation and growing tissue within the dynamic environment of a living organism for extended implantation. The stretch sensor improves upon a previous iteration, the flexible pulsation sensor (FPS) that was found to have a number of failure points during chronic implantation. The FPS was described at least in (1) U.S. Pat. No. 10,694,999, (2) U.S. Pat. No. 11,576,612, and (3) US 2024/0023821, which are hereby incorporated by reference in their entirety.

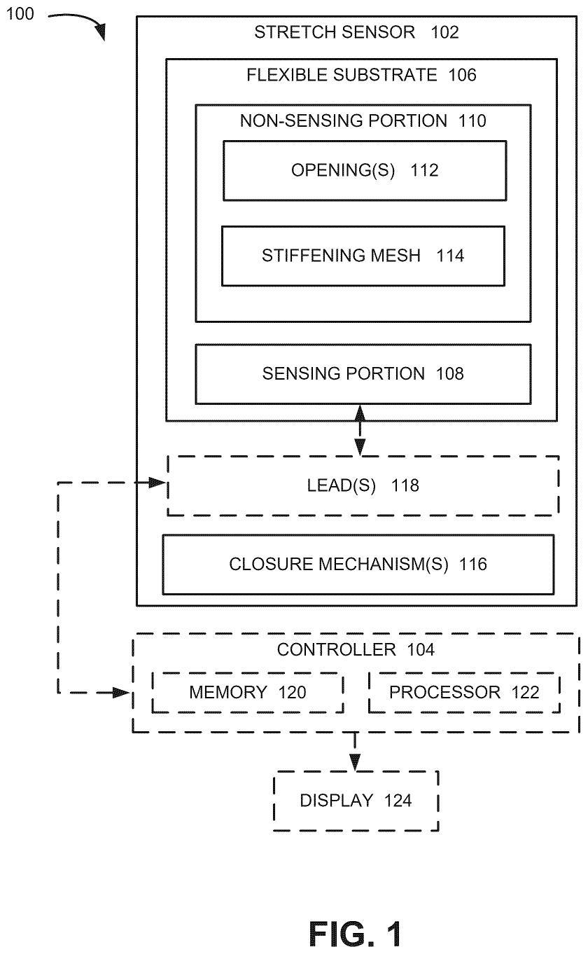

FIG. 1 shows a block diagram of a system 100 that includes a stretch sensor 102 in communication with a controller 104. The system 100 can continuously monitor blood pressure and/or blood flow of a patient with an implanted stretch sensor 102 (the stretch sensor may be referred to herein as “sensor”). The stretch sensor 102 performs more favorably during chronic implantation compared to previous iterations of the FPS. Like the previous iterations of the FPS, the stretch sensor 102 can utilize the piezoresistive effect to transduce mechanical deformation into a change in resistance that can be measured by a connected controller. The stretch sensor 102 can include a flexible substrate 106 that can be wrapped around at least a portion of an artery. The stretch sensor 102 can have a sensing portion 108 in an intermediate portion of the flexible substrate 106 and a non-sensing portion 110 that can be split between opposite ends of the flexible substrate. The sensing portion 108 can detect changes in a diameter of an artery a sensor is wrapped around. The sensing portion 108 can include a piezoresistive material that includes conductive particles (such as carbon black nanoparticles and carbon nanotubes) suspended in a polymer (such as carbon black-polydimethylsiloxane (CB-PDMS)).

However, unlike previous iterations of the FPS, the stretch sensor 102 is both strong enough to withstand forces acting upon it from an artery (that the stretch sensor is wrapped at least partially around) and other forces (e.g., bodily forces, external forces, or the like) and compliant enough to deform in response to minute changes in internal pressures within the artery such that its measurements of mechanical deformation can be accurate. These advantages of the stretch sensor 102 are evident in the non-sensing portion 110, which can include one or more openings “opening(s)” 112, also referred to as holes) through the flexible substrate 106. At least one of each of the opening(s) 112 can be positioned on each end of the opposite ends of the flexible substrate 106. When an end includes more than one opening(s) 112, then each of the openings can be longitudinally positioned in a line to allow for re-sizing of the stretch sensor for arteries of differing diameters. In some instances, the opening(s) 112 can be made during implantation of the stretch sensor 106 to precisely fit a specific artery. The opening(s) can be any size and/or shape that can withstand tear forces from closure but are shown throughout as having an elliptical cross-section for ease of illustration. It is noted that an elliptical shape opening is known as beneficial for reducing tear forces.

The non-sensing portion 110 can also include a stiffening mesh 114 around at least each of the opening(s) 112 to improve the tear strength of the non-sensing portion of the sensor. The stiffening mesh can be a nylon mesh, a surgical mesh, and/or another stiffening material that can be embedded into the flexible substrate 106. It is noted that in chronic implantation of the previous FPS the closure regions were a substantial failure point, with sutures failing and/or openings tearing over prolonged implantation and uses time. The stretch sensor 102 also includes one or more closure mechanisms “closure mechanism(s)” 116 that can connect the opposite ends of the flexible substrate 106 via the opening(s) on each of the opposite ends of the flexible substrate. The closure mechanism(s) 116 can be, for instance, snap-rivet closure(s), suture(s), screw-type closure(s), adhesive(s), and/or the like that can be biocompatible and low profile (e.g., 3 mm-10 mm outer diameter, 0.5 mm-3 mm diameter shank, or the like) so as to not disturb bodily tissues and/or cause significant fibrotic growth and/or autonomic activation.

The stretch sensor 102, in some instances, can have at least one lead “lead(s)” 118 that can interface with the sensing portion 108 to transmit the detected changes in the diameter of the artery (in the form of electrical resistance changes) to the controller 104. In other instance(s), not shown, the leads can be replaced with a transceiver to facilitate wireless communication between the sensing portion 108 and the controller 104. When the data is received from the stretch sensor 102 by wired and/or wireless communication, the controller 104 can execute instructions to determine at least one measurement related to arterial pressure based on the data and then output the at least one measurement related to the arterial pressure. The at least one measurement related to the arterial pressure can be blood pressure, blood flow, and/or another related cardiovascular variability parameter. The controller 104 can include a non-transitory memory, “memory” 120 for storing instructions, and optionally data and/or output measurements. The controller 104 can include a processor 122 that can execute the instructions. The memory 120 and the processor 122 can be embodied as separate devices and/or a single device such as a microprocessor with both capabilities.

In some instances, the controller 104 can execute instructions to output a blood pressure reading. The controller 104 can receive data related to strain of the sensing portion 108 of the stretch sensor 102. The data can be electrical resistance at a time of the piezoresistive material in response to deformation from changes in arterial wall pressure at the time. A baseline of the electrical resistance can be pre-determined (e.g., during calibration) so changes can be noted. The data output by the stretch sensor 102 is linear compared to an internal arterial pressure and is directly proportional to a change in the internal pressure. The controller 104 can, for each time, determine an internal pressure of the artery (e.g., arterial pressure) based on the data. The controller can convert the resistance measurements into arbitrary units that can then be converted to blood pressure measurements based on the calibration of the stretch sensor 102. The controller 104 can then output a blood pressure reading. In some instances, the controller 104 can also determine one or more other cardiovascular variability parameters for the patient based on at least one blood pressure reading, sometimes including other physiological measurement(s) of the patient. For instance, the controller 104 can determine and output an estimation of volumetric blood flow rate based on a plurality of blood pressure readings over time

The system 100 can also include a display 124 that can be in communication (wired and/or wireless) with the controller 104 to at least display a visualization the measurements and/or the data recorded by the stretch sensor 102 (e.g., numerically, graphically, pictorially, etc.) related to the arterial pressure to a user and/or an associated medical professional. The display 104 may additionally and/or alternatively include an auditory and/or a haptic measurement for providing audible and/or tactile versions of the measurements. In some instances, the system 100 can provide a warning to a patient, an associated medical professional, and/or an emergency service and/or contact when a measurement at a time or a trend over a period of time are outside one or more limits (high and/or low) set for the patient by a medical professional.

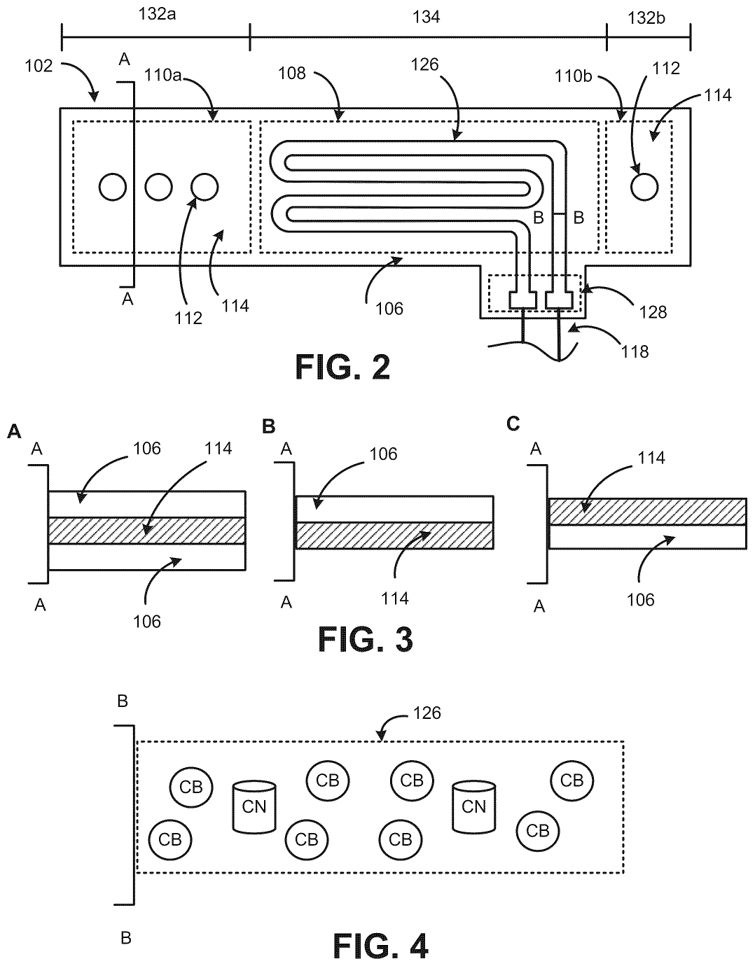

FIG. 2 shows an example of an inner side (that would face an artery) of the sensor 102 in a flat configuration (in greater detail). The flexible substrate 106 has opposite ends 132a and 132 b and an intermediate portion 134. Each opposite end 132a and 132b of the flexible substrate 106 can include a portion of the non-sensing portion 110a and 110b, while the intermediate portion 134 can include sensing portion 108. As shown, a portion of the non-sensing portion 110a can include three openings 112 through the substrate and another portion of the non-sensing portion 110b can include a single opening. It should be understood that these are only examples and each non-sensing portion 110a and 110b can include any number of openings 112 one or greater in any shape/size and/or position. The non-sensing-portions 110a and 110b can include embedded stiffening mesh 114 (described in more detail in FIG. 3 with respect to cut line A-A). The flexible substrate comprises a soft, biocompatible material that provides an increased compliance to deformation of a wall of the artery. When in use, the flexible substrate 106 can be bent so that at least one opening 112 on each end 132a and 132 b can overlap in some manner and a closure mechanism (not shown in FIG. 2) can connect the two ends through the respective openings.

The sensing portion 108 can include at least one piezoresistive material that can be arranged in a resistive pattern 126. It should be understood that only an exemplary pattern is shown and any other pattern capable of stretching in response to changes in artery diameter is included herein. The piezoresistive material is described in more detail with respect to cut-line B-B in FIG. 4. The sensing portion 108 can be connected with one or more electrically conductive leads 118 (shown as two) at an interface 128. The interface 128 can include a room-temperature-vulcanizing (RTV) silicone material that can interface between the at least one lead and the sensing portion to secure the at least one lead to the flexible substrate with greater strength than previous FPS connections. It is noted that the interface connections are another point of failure during of the previous FPS during chronic implantation as the connections broke and/or the leads failed, causing degradation of data collection abilities. The configuration of the stretch sensor 106 reduces and/or eliminates this point of failure of the previous FPS.

FIG. 3 shows example configurations of the non-sensing portion 110a of the sensor 102 of FIG. 2 at cut-line A-A. The different configurations show different example positions of the stiffening mesh that are possible but not limiting. It should be understood that non-sensing portion 110b can be similar and/or identical in composition. FIG. 3, element A shows an example of the stiffening mesh 114 embedded within layers of the flexible substrate 106 material. FIG. 3, element B shows an example of the stiffening mesh 114 embedded as a layer on a bottom of the flexible substrate 106 material. And, FIG. 3, element C shows an example of the stiffening mesh 114 embedded as a layer on top of the flexible substrate 106 material. The stiffening mesh 114 can be added to the entire width and length of the non-sensing portions 110a and 110b. The stiffening mesh 114 can be, for instance, a nylon mesh formed of 90% nylon and 10% elastane (each plus or minus 10%, 5%, 2% or less, or the like). In other instances, the stiffening mesh 114 can be a surgical mesh and/or any other compositions that can stiffen the non-sensing portions (e.g., 110a and 110b) compared to the sensing portion. For example, the flexible substrate 106 can be a material that can have a Young's Modulus of approximately 40 kPa-45 kPa (e.g., 43.3 kPa) and the stiffening mesh 114 can have any reasonable higher Young's Modulus (e.g., that does not negatively affect the ability of the sensing portion 108 in collecting data related to changes in artery diameter).

The resistive pattern 126 of sensor 102 in FIG. 2 includes a strain gauge that includes piezoresistive materials and can exhibit changes in electrical resistance when subjected to mechanical deformation from pressure within an artery. FIG. 4 shows an illustrative example of a cut-line B-B through a portion of the piezoresistive material of the resistive pattern 126. A strain gauge can be characterized by a gauge factor that is directly related to the strain gauge's sensitivity. The higher the gauge factor the greater the sensitivity of the strain gauge. The gauge factor is the ratio of the relative change in electrical resistance to the mechanical strain experienced by the gauge. Mathematically, gauge factor (GF) is expressed as:

$$\frac{Δ R}{{R}_{0} ϵ} = \frac{Δ R}{{R}_{0} ( \frac{δ}{{L}_{0}} )} = \frac{Δ R \cdot {L}_{0}}{{R}_{0} δ}$$

where ΔR is the change in resistance, δ is the change in length, R₀ is the initial resistance of the gauge, L₀ is the initial length, and ε is the applied strain. The gauge factor quantifies how much the electrical resistance of the strain gauge changes in response to a given amount of mechanical strain. The piezoresistive material and stretch sensor (e.g., 102) described herein has a gauge factor approximately 15 times greater than the previous FPS. However, the gauge factor can be any value from 5-45 times greater than the previous FPS.

The piezoresistive material of the resistive pattern 126 described herein includes a piezoresistive composite material known as carbon black polydimethylsiloxane (CB-PDMS) that includes carbon black nanoparticles (CB) and carbon nanotubes (CN) suspended in the PDMS. The piezoresistive material can include, for instance, approximately 80% carbon black nanoparticles and 20% carbon nanotubes (plus or minus 10%, 5%, 2%, or the like). As arterial pulsations influence the sensor's shape (cause stress/deformation), the piezoresistance of the CB-PDMS enable it to detect changes in diameter. The material composition directly impacts sensor performance and overall flexibility. The combination of carbon black particles with multi-walled carbon nanotubes greatly improved sensor performance compared to the prior FPS that used only carbon black or only carbon nanotubes. The combination of carbon black with carbon nanotubes allows a much lower concentration of carbon nanotubes while still allowing for conductive (resistive) sensor material. This improves elasticity, mixing/processing of the material, and most importantly, sensor gauge factor/sensitivity (e.g., by approximately 15 times the prior FPS in this example).

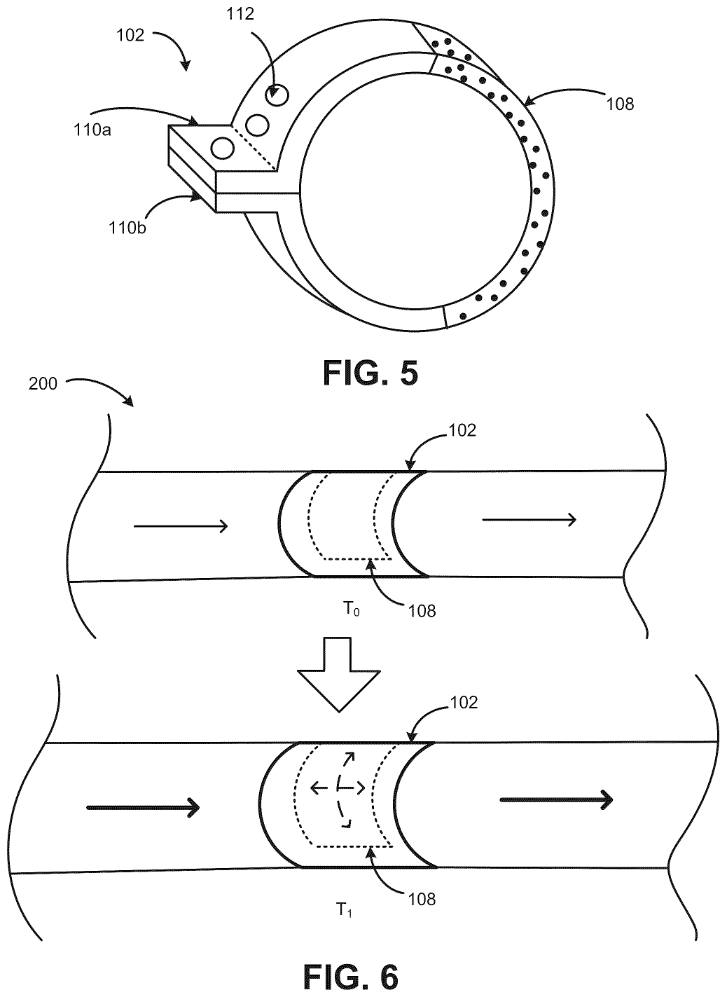

FIG. 5 shows a profile view of the sensor 102 in a wrapped configuration with a “duckbill” closure. The artery is not shown for ease of illustration, but the flexible substrate 106 can wrap around at least the portion of the artery (as shown in FIG. 6) with the sensing portion 108 in the intermediate portion of the flexible substrate at least partially encircling the at least the portion of the artery and the non-sensing portion 110a and 110b on the opposite ends of the flexible substrate closing around the at least the portion of the artery. The non-sensing portion 110a on one of the opposite ends of the flexible substrate 106, as shown, can overlap the non-sensing portion 110b on another of the opposite ends of the flexible substrate such that openings 112 on each of the opposite ends of the flexible substrate overlap and the one or more closure mechanisms (116, not shown in FIG. 5) connect through the openings to secure the flexible substrate around at least the portion of the artery. In one instance, the closure regions of the non-sensing portions can protrude outwards from the artery in a “duckbill” that means the portions of the closure mechanism(s) 116 do not contact the artery and/or interfere with movement of the artery.

The flexible substrate (e.g., 106) that comprises the base of the sensor 102 can be a soft, biocompatible material that provides an increased compliance compared to deformation of a wall of an artery. The increased compliance can inhibit the activation of growth factors (such as TGF β1) that can lead to fibrotic activation. The increased compliance can also allow for the stretch sensor to be more sensitive to deformation of the wall of the artery. The flexible substrate material can be, for instance, a platinum-catalyzed silicone material such as Ecoflex™ 00-10 that has a low elastic modulus and is biocompatible. Such a flexible substrate material can increase toughness to improve longevity in-vivo for chronic implantation and allow for more energy absorption from distension of the arterial wall (e.g., greater transduction of the sensor) which increases sensitivity compared to the previous FPS. The previous material used had a higher durometer of Shore A 00-10 hardness. Thus, the total displacement before failure increased from 35 mm total to about 10 5 mm before failure (3× increase) and the force gradient (can be converted to Young's Modulus by normalizing by the cross section of the sensor) decreased approximately 12×.

FIG. 6 shows an illustration 200 of how an example sensor 102 can stretch due to the flow and pressure of blood in an artery. The sensor 102 can be wrapped around at least a portion of an artery. Changes in the artery's diameter can cause deformation in the sensing portion 108 of the sensor 102, particularly the resistive pattern that forms the strain gauge as discussed in greater detail with respect to FIG. 2. Changes in the deformation of the sensor can be measured as changes in resistance in the strain gauge. The resistance can be used to determine blood pressure and/or blood flow based on linear relationship and pre-calibration. The sensor 102, and/or at least the sensing portion 108, can have a compliance that is the same as or more than the artery it is wrapped around to allow the sensor to be sufficiently sensitive to deformation in the artery wall due to pressure. As shown, at time T₀ the artery is in a non-distended state (e.g., blood pressure is at a trough of a blood pressure waveform) and at time T₁ the blood pressure and/or flow has increased, causing the artery walls to expand and the sensor to deform (stretch) in response.

The sensor 102 includes materials with a reduced elastic modulus that allows for higher displacement/strain of the sensor at a given arterial pressure. This allows for increased compliance of the sensor to deformation of the arterial wall (increased sensitivity), while minimizing the effects of reaction forces on arterial blood pressure. Previous materials used were of a higher durometer, such that the artery did not appropriately stretch them. By switching to a durometer of Shore A 00-10 hardness, the sensor 102 is inherently more elastic than the artery and achieves maximal strain during the cardiac cycle. In comparison to the previous FPS, the total displacement before failure increased from 35 mm total to about 105 mm before failure (3× increase). The force gradient (which can be converted to Young's Modulus by normalizing by the cross section of the sensor) decreased approximately 12× (but the decrease can be any value between 5-45×). The reduced elastic modulus also makes the sensor tougher and more sensitive to the effects of vascular tone on arterial distension than previous FPS because the elastic modulus allows for more energy absorption from distention of the arterial wall (greater transduction to the sensor).

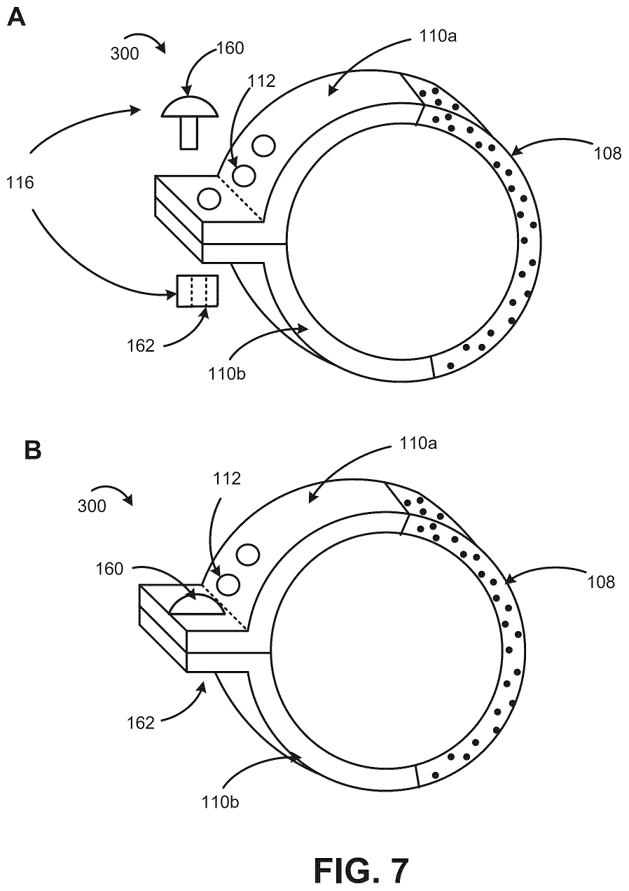

FIG. 7 shows an example of a stretch sensor 300 that is designed with ends that meet outside the encirclement of the artery in a “duckbill” configuration. FIG. 7, element A shows an example two-part closure mechanism that can be pushed through two overlapping holes 112 in the “duckbill” portion of the sensor 300. The closure mechanism 116 is an example two-part closure mechanism comprising a male portion 160 and a female portion 162 that fastens when the male portion is at least partially pushed into the female portion. The mechanism of fastening can be snap-rivet (e.g., a portion of the male portion 160 expands and cannot be pulled back through the female portion 162), suction (e.g., the female portion tightens on the male portion), a screw mechanism, or another mechanical means that locks at least a portion of the male part in the female part. The substrate 106 bound portion of the example sensor 300 can include the two-non sensing portions 110a and 110b bracketing an intermediate sensing portion 108. Each of the non-sensing portions 110a and 110b include at least one hole 112 (shown as 3 holes in 110a and with holes in 110b not visible in these views) that the male portion of the closure mechanism 116 can be pushed through to fasten the sensor around at least a portion of an artery (e.g., around a circumference of an artery, artery not shown for ease of illustration and description). FIG. 7, element B, shows the example sensor 400 completely fastened with a snap-rivet closure securing the opposite ends. The closure mechanism 116 is fastened through an opening (e.g., 112) in each non-sensing portion 110a and 110b. The snap-rivet closure can at least partially prevent tearing of the non-sensing portion 108 due to stresses on the example stretch sensor 300. In this example, the male portion (e.g., 160) of the closure mechanism has a head remaining visible (although may be flatter/differently shaped than illustrated) and a portion that protrudes through the overlapping holes of the two non-sensing portions 110a and 110b and then into the female part 162 where it catches (not visible within and beneath the duckbill). While shown with the male portion 160 on the top and the female portion 162 on the bottom, this can be reversed.

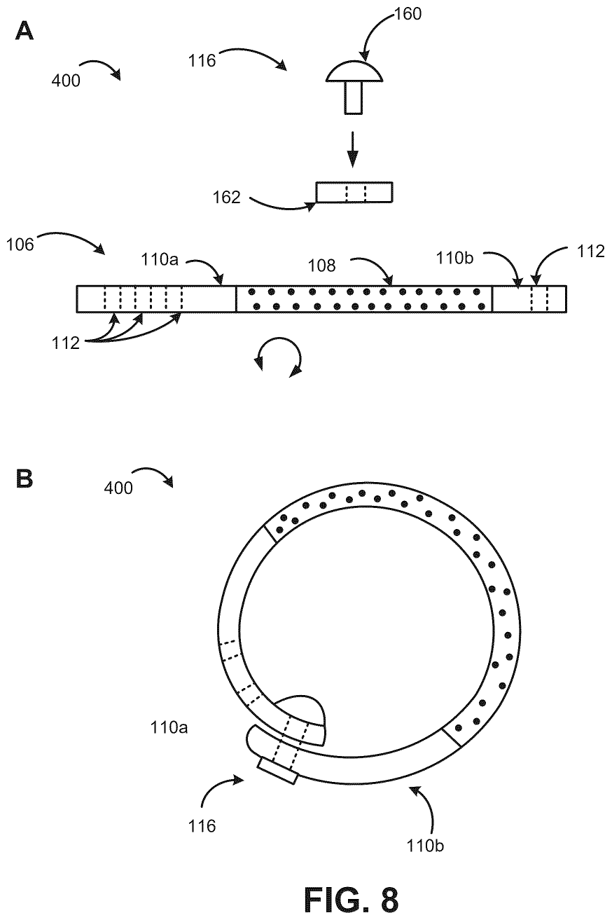

FIG. 8 shows another example of a stretch sensor 400 that is designed for the ends 110a and 110b to overlap in-line with the encirclement of the artery. FIG. 8, element A, shows the substrate 106 bound portion of the sensor 400 separate from a closure mechanism 116 of the sensor. The closure mechanism 116 is an example two-part closure mechanism comprising a male portion 160 and a female portion 162 that fastens when the male portion is at least partially pushed into the female portion. The mechanism of fastening can be snap-rivet (e.g., a portion of the male portion 160 expands and cannot be pulled back through the female portion 162), suction (e.g., the female portion tightens on the male portion), a screw mechanism, or another mechanical means that locks at least a portion of the male part in the female part. The substrate 106 bound portion of the example sensor 400 can include the two-non sensing portions 110a and 110b bracketing an intermediate sensing portion 108. Each of the non-sensing portions 110a and 110b include at least one hole 112 (shown as 3 holes in 110a and 1 holes in 110b) that the male portion of the closure mechanism 116 can be pushed through to fasten the sensor around at least a portion of an artery (e.g., around a circumference of an artery). FIG. 8, element B shows the example sensor 400 in a wrapped configuration (artery not shown for ease of illustration and description) where the ends of the two non-sensing portions 110a and 110b overlap in line with the wrapping and the closure mechanism 116 being a snap-rivet closure that is fastened through a hole (e.g., 112) in each non-sensing portion. The snap-rivet closure can at least partially prevent tearing of the non-sensing portion 108 due to stresses on the example stretch sensor 400. In this example, the male portion (e.g., 160) of the closure mechanism protrudes through the overlapping holes of the two non-sensing portions 110a and 110b and then into the female part where it catches. While shown with the male portion on the inside and the female portion on the outside, this can be reversed. This closure mechanism 116 of the stretch sensor 102 is an improvement over previous FPS. It should be understood that the snap-rivet (or a different type of mechanical closure with operating mechanism similar to that of the snap-rivet) does not preclude the use of sutures or other non-mechanical means (e.g., in other holes, faces, etc. of the sensor 102). In some instances, the closure can be via the snap rivet alone. In other instances, the snap rivet can be used in connection and/or combination with sutures and/or adhesive. The sensor 102 utilizes the snap rivet as the primary means to remain closed and to continue sensing fidelity while implanted.

### IV. Methods

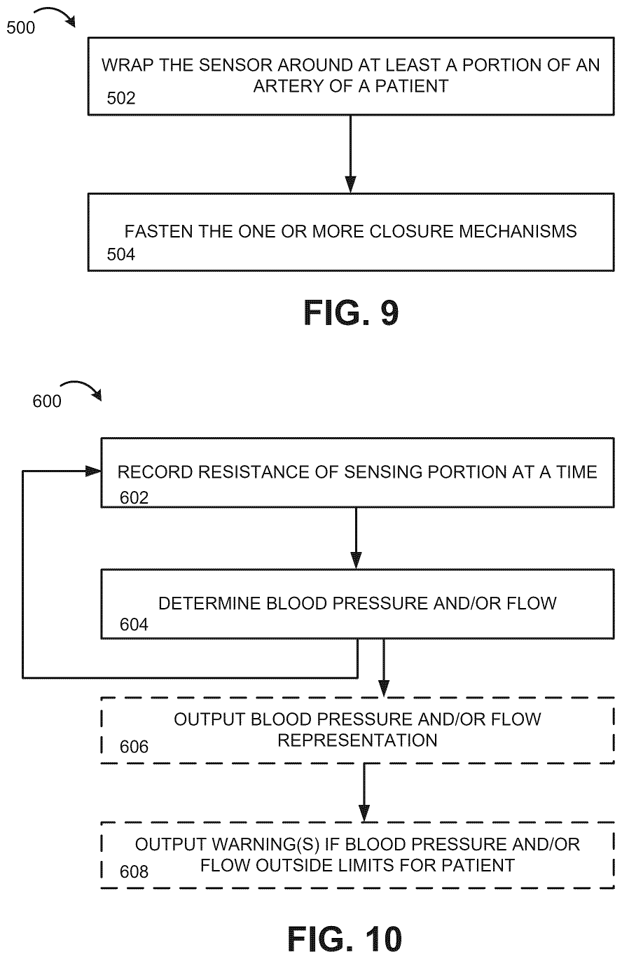

Another aspect of the present disclosure can include example methods for implantation 500, continuous monitoring 600, and fabrication 700 of the stretch sensor (e.g., 102 of FIG. 1 and modified in FIGS. 2-8). The methods 500, 600, and 700 are illustrated as processes flow diagrams with flowchart illustrations that can be implemented by one or more components of the stretch sensor, an associated controller, or the like, as shown in FIG. 1. For purposes of simplicity, the methods 500-700 of FIGS. 9-11 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement methods 500, 600, and 700.

FIG. 9 shows an example method 500 for implanting the sensor in a patient. While not shown, the patient can undergo a surgery to expose/reach at least one artery—the surgery can be, for example, a traditional surgery and/or a non-invasive surgery. At 502, the sensor can be wrapped around at least a portion of a target artery that has been exposed/reached. The sensor ends can overlap, meet, or the like when wrapped. The sensor can, for instance, be wrapped around a carotid artery. Additionally, it should be noted that more than one sensor can be positioned on more than one artery (e.g., bilateral placement and/or placement above and/or below a possible stenosis, known graft, or the like. The sensor can be wrapped such that the sensing portion encircles and/or at least partially encircles a portion of the artery such that the sensing portion can react to changes in the diameter of the artery. The sensor can be wrapped such that the blood flow in the artery is not affected by the sensor, but tightly enough to detect even slight changes in diameter noting that the sensor can have a compliance that is equal to or less than the compliance of the arterial wall. For example, the wrapping can be as shown in FIG. 7, elements a and b with a “duckbill closure area” or FIG. 8, element b with an “inline closure area”. The wrapping can be done by a surgeon with one or more instruments, by hand, and/or using a surgical robot.

At 504, one or more closure mechanisms can be fasted to keep the sensor wrapped around the artery. The one or more closure mechanisms can be a snap-rivet closure (or any different type of mechanical closure mechanism that works mechanically similarly to the snap-rivet). In some instances, other closure mechanisms can be used with the snap-rivet, including one or more sutures, an adhesive (such as a UV curable, biocompatible adhesive), or the like. If the closure mechanism requires a hole (such as a snap rivet or other type of mechanical fastener; a suture may also work in secondary holes), then hole(s) can be made on each end of the wrapped sensor that connect. If the closure mechanism is a snap rivet, then the holes can be pre-punched into each end of the sensor and measured to fit the artery being wrapped. A protruding part of the male part of the snap-rivet fastener can be pushed through the holes and then into the female part of the snap-rivet fastener, where the protrusion catches and holds. The closure mechanism can be slim enough and biocompatible such that it does not cause excess fibrotic growth and/or interfere with measurements. It is important to note that the areas used for fastening are stiffened in comparison to the rest of the sensor to prevent tearing around the holes/closure mechanisms.

FIG. 10 shows an example method 600 for using the implanted stretch sensor for continuous monitoring of at least blood pressure and/or blood flow. Method 600 can include a controller in communication (wired and/or wireless) with the sensor that can perform one or more of the steps described below.

At 602, a resistance of the sensing portion of the stretch sensor can be recorded at a time (e.g., by the sensor itself or a controller in communication with the sensor and receiving data from the sensor). The sensor (e.g., 102) can include a strain gauge that includes piezoresistive material (e.g., carbon black nanoparticles and carbon nanotubes embedded in PDMS). The strain gauge exhibit changes in electrical resistance when subjected to mechanical deformation from pressure within the artery as the diameter of the artery changes due to blood movement. The gauge factor of a strain gauge characterizes the strain gauge's sensitivity, the higher the gauge factor the more sensitive the gauge, and the gauge factor quantifies how much the electrical resistance of the strain gauge changes in response to a given amount of mechanical strain. It should be noted that the sensor described herein (comprising 80% carbon black nanoparticles and 20% carbon nanotubes, or the like, and the mechanical adjustments also describe above) has a gauge factor approximately 15 times greater than the previous iteration which leads to improved resolution and accuracy.

At 604, an arterial pressure waveform, blood pressure, and/or blood flow can be determined (e.g., by the controller 104). The determination can be made by comparing the resistance recorded at the time to a resistance previously noted for a baseline pressure (e.g., during troughs in the blood pressure waveform). A sensor can be calibrated such that the system can map a given change in resistance to a known blood pressure and/or multiple changes over time to an arterial pressure waveform and/or blood pressure waveform. Additionally, the stress sensor is sensitive to changes in pressure within the artery rather than direct measurements of blood flow (e.g., does not directly measure volumetric flow rate). However, in situations such as arterial stenosis (artery narrowing) the resistance to blood flow increases and the stress sensor can detect changes in the arterial wall deformation. Additionally, blood flow and pressure are linked and flow dynamics can be estimated (e.g., by the controller) from the recorded values. Blood flow is typically represented as the volume of blood passing through a specific point or cross-sectional rea per unit time. Blood flow is directly proportional to the pressure difference across the venous return and variations in arterial pressure create a driving force for blood flow. The estimation can be based on a known correlation between the sensor's pressure-related measurements and volumetric flow. For continuous monitoring, the steps 602 and 604 can repeat at a given sampling frequency until the power source runs out, the sensor is made to stop recording, or the like.

Optionally, at 606, the determined blood pressure and/or blood flow representation can be output to a memory (e.g., memory 122) and/or visually (e.g., numerically, graphically, etc.), audibly, and/or tactically (e.g., to a display 124, speaker, and/or haptic) to the patient and/or a medical professional associated with the professional. The output can be set to automatically occur based on time (e.g., every 30 minutes, every hours, or the like), continuous (at the recording frequency), and/or when manually queried by a patient and/or medical professional. Optionally (additionally or alternatively to 606), at 608, a warning can be output. The warning can be output visually (e.g., numerically, graphically, etc.), audibly, and/or tactically (e.g., to a display 124, speaker, and/or haptic. The warning can be output when the blood pressure and/or blood flow is above a limit (e.g., elevated/low blood pressure spike) and/or a limit for an amount of time (e.g., elevated/low blood pressure for X amount of time triggers a warning) set by a medical professional for the specific patient (e.g., based on patient demographic factors, disease status, or the like). The warning can be output to the patient, the medical professional, and/or emergency contact (lay person or emergency service).

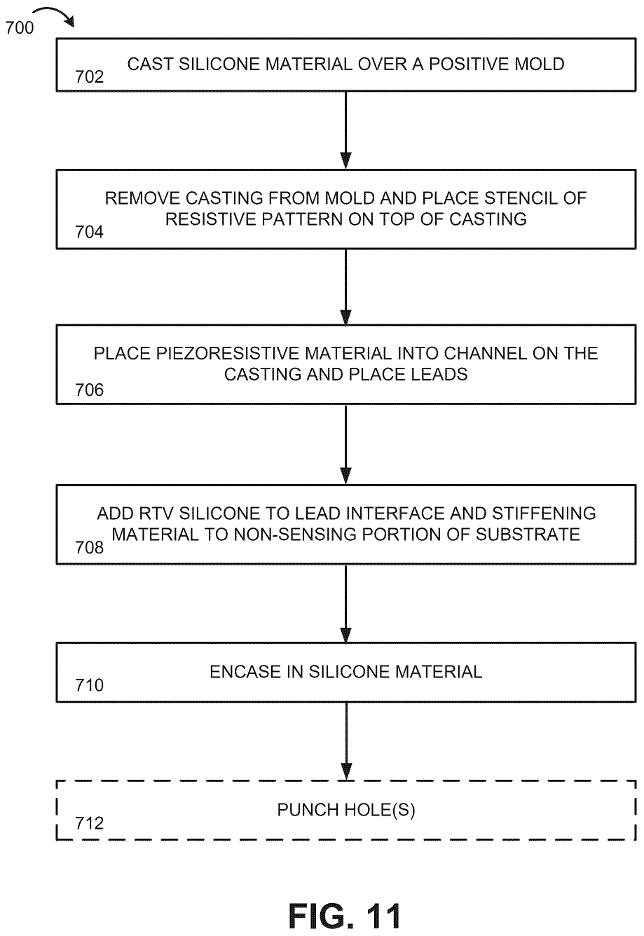

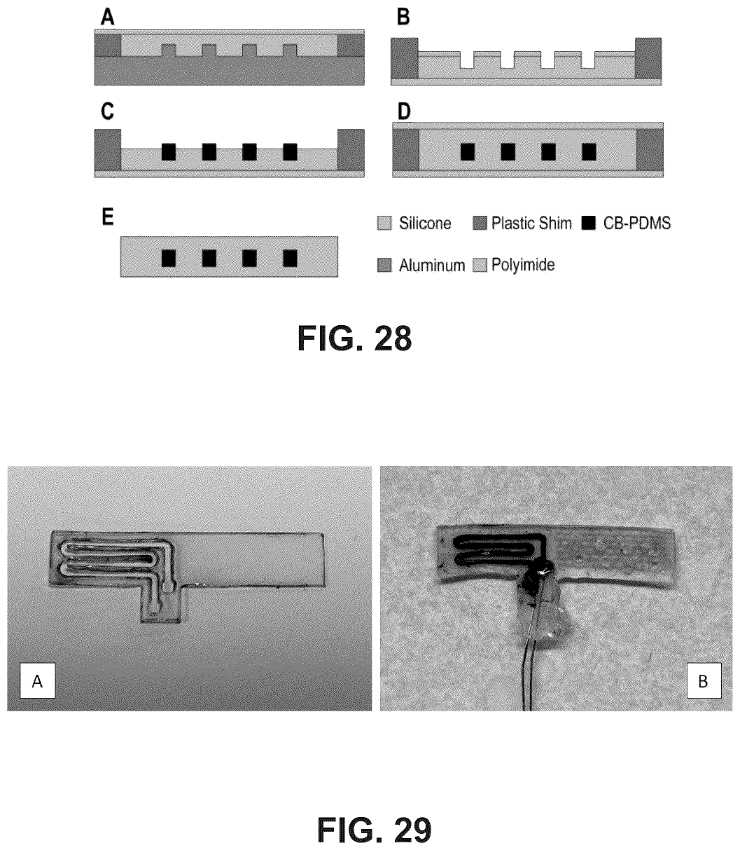

FIG. 11 shows an example method 700 for fabricating the stretch sensor. FIG. 28 of the experimental shows a visual representation of steps associated with method 700. At 702, the substrate silicone material (e.g., Ecoflex™ 00-10) can be cast over a positive mold of the sensor to create a channel in the desired pattern for the resistive material. At 704, the casting made of the substrate silicone material can be removed from the mold and a stencil of the resistive pattern can be placed on top of the casting. At 706, the CB-PDMS composite material can be placed into the channel in the casting and Pt(0)Ir leads can be embedded into at least a portion of the CB-PDMS composite material to establish electrical contact. At 708 RTV silicone can be added to the lead interface area and a stiffening material (e.g., a stiffening mesh, such as a surgical mesh or a nylon mesh) can be added to at least part of the non-sensing portions of the substrate. At 710, the sensor components (as described above-substrate, resistive layer, and lead connections) can be encased in a final layer of the silicone material (e.g., Ecoflex™ 00-10) to seal the sensor. Optionally, at 712 at least two holes (at least one on each end of the substrate) can be punched out of the silicone material (including the embedded stiffening mesh) for securing with the closure mechanism(s) such as the snap-rivets described above. In another instance, the holes can be formed just before or during implantation using a sharp object such as a needle. In a further instance, no holes are necessary and an adhesive such as a UV curable, biocompatible adhesive can be used to join together the two ends. It should be understood that the “holes” do need to be created in the silicone material but need not be created according to 712.

The CB-PDMS composite material can itself be fabricated with the following steps. Carbon black granules can be ground (e.g., with a mortar and pestle) to a desired size and an amount of the carbon black nanoparticles can then be positioned with another amount of carbon nanotubes into an empty container (e.g., a beaker). For example, the ratio can be 80% carbon black granules (e.g., nanoparticles) to 20% carbon nanotubes. An amount (e.g., 10 g) of a carrier fluid (e.g., Vertrel) can be added to the container with the carbon black granules and the carbon nanotubes. The mixture can be sonicated for a time (e.g., 20 minutes at 500 W power, 20 KHz frequency, and a 20% duty cycle). Then an amount of one silicone material (e.g., Ecoflex™ 00-10 Part A) and another silicone material (Ecoflex™ 00-10 Part B) can be added to the container and another amount of carrier fluid (e.g., 10 additional g of Vertrel). The mixture can then be mixed (e.g., by hand) for approximately 2-3 minutes. The mixture can then be sonicated again for another time (e.g., 20 minutes at 500 W power, 20 KHz frequency, and a 20% duty cycle). The resultant mixture can then be powered into a tray (e.g., an aluminum tray) and magnetically stirred until the mixture becomes too viscous to continue stirring (approximately 20 minutes). Finally, the tray can be transferred to a vacuum desiccator and degassed until about 3% (e.g., between 2.5% and 3.5%, between 2.75% and 3.25%, or the like) of the carrier fluid (e.g., the Vertrel) remains (approximately 20 to 20 minutes). The final product can be used as the resistive layer in the fabrication of the stretch sensor.

### V. Experimental

The following experiments demonstrate uses and construction of the stretch sensor described above. The findings of these experiments indicate that the implanted stretch sensor can be constructed differently from standard FPS sensors and perform continuous blood pressure/flow monitoring more accurately than standard FPS sensors.

### A. In Vivo Sustainability

The standard FPS has been shown to be sustainable in vivo. However, the standard FPS has been shown to be lacking due to mechanical failures and signal degradation. A new stretch sensor should have similar in vivo properties to the standard FPS but improve areas where the standard FPS is lacking.

### 1. Pig Studies of the Standard FPS

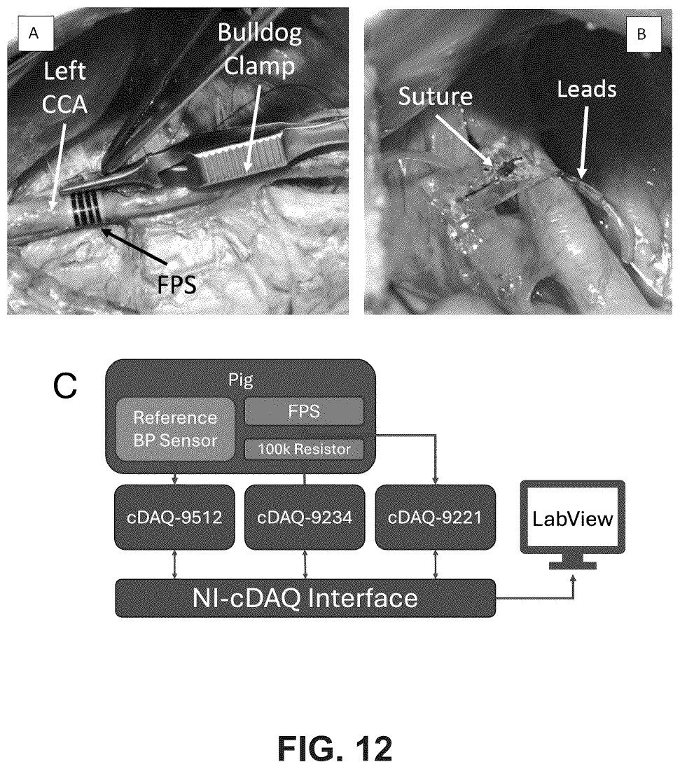

For in vivo characterization of the standard FPS sensor, the standard FPS was chronically implanted into growing female pigs (n=3) weighing between 30 kg and 40 kg at the time of implant see FIG. 12, elements A and B. Standard FPS were implanted bilaterally around the common carotid artery, ranging in size from 4 mm-6 mm. This placement allowed for monitoring of blood pressure and changes in the hemodynamic waveform while animals were awake and asleep. The implantation surgery was conducted with care to ensure minimal disruption to the carotid artery. Readout leads from the sensor were tunneled to an exit site at the back of the neck. Leads were also covered by a wound dressing, shirt, and rip-proof jacket. Post-implantation, continuous data acquisition was carried out over 71, 108, and 123 days for each pig respectively, under both anesthetic and ambulatory conditions. Pigs gained 54-70 kg over the duration of their implantation.

To measure the signal from the standard FPS, leads were connected in a quarter-bridge configuration to an instrumentation system using National Instruments CDAQ-9221, cDAQ-9512 and cDAQ-9234 modules, controlled by a LabVIEW program, as shown in FIG. 12, element C. A lowpass median filter was applied as a smoothing filter to remove high frequency noise from the signal.

During the implantation surgery, data from an arterial catheter was recorded as a gold standard measurement while FPS data were recorded. This simultaneous recording allowed for a direct assessment of the standard FPS's performance in capturing cardiovascular events in comparison to the established measurements provided by the arterial catheter. Just after implant, the standard FPS exhibited good signal fidelity, enabling the capture of specific cardiovascular events, particularly the dicrotic notch. The dicrotic notch was a distinct notch or small dip that appears on the descending portion of the arterial pulse wave in a blood pressure waveform which occurs just after the closure of the aortic valve. The notch is associated with the brief interruption of the smooth downward flow of blood in the aorta due to the closure of the aortic valve.

Over the course of several months of implant, the standard FPS exhibited significant data quality degeneration. Notably, the full scale of the signal decreased greatly, indicating either a decrease in the gauge factor of the sensor, or a reduction of force transduced through the sensor. Additionally, the recorded waveform no longer represented a typical cardiac signal, though systolic peaks could still be discerned. This deterioration in signal quality raised concerns about the standard FPS's long-term reliability and prompted the need for further investigation into the underlying factors contributing to the decline.

In several cases, the leads at the interface between the Pt—Ir wires, and the CB-PDMS became dislodged, or otherwise disconnected from the externalized leads. Gentle pressure on the skin above the standard FPS restored electrical contact in most cases. While this solution worked during measurement under anesthesia, the unrestricted nature of ambulatory measurements presented a challenge. In one case, the suture closure mechanism used to attach the standard FPS to the artery failed towards the end of the study, resulting in inaccurate measurements of arterial distention.

Overall, the standard FPS performed fairly well, with the sensor materials enduring over 4 months of implantation in changing biological environments. Salient issues with the standard FPS pertained to the mechanical detachment of the leads and failure of the suture mechanism. These mechanical challenges pose significant concerns for the standard FPS's reliability and long-term performance. Addressing these issues requires a thorough examination and potential redesign of the mechanical components to enhance the standard FPS's durability and secure placement, ensuring consistent and accurate monitoring in various applications.

### 2. Revising Design Due to Challenges of the Standard FPS Sensor

Due to the challenges discussed in section 1, the standard FPS sensor needed to be re-designed. The revised sensor design was also subjected to in vivo characterization and while the signal amplitude itself demonstrates high fidelity of the underlying pulsation, as evident by the low frequency oscillations correlating to respiration, the quality of the signal itself still falls short of expectations. Indeed, slight perturbations of the lead interface causes sudden jumps in the voltage as the leads momentarily lose connection.

The presence of artifacts significantly undermines the capacity of the re-designed FPS sensor to establish an effective correlation with units of blood pressure. To achieve meaningful and clinically relevant results, it becomes imperative to identify and mitigate these artifacts. The bond between PDMS and metals are inherently weak, which is a major factor in the appearance of these artifacts. In microfluidic manufacturing, several bonding techniques are used to increase the bond strength between PDMS and other polymers/ceramics. Chemical and non-chemical procedures have also been investigated to attempt to bond PDMS to metal substances. Some of these tactics were employed to ensure a tough and robust connection to the CB-PDMS.

### 3. Pressure Calibration

Correlation of the arbitrary units (AU) of the FPS to units of blood pressure is an important consideration in the clinical applicability of the sensor. Assuming that the sensor is linearly associated to strain (and later simulated with Finite Element Analysis), a linear transformation of the form:

$$A \vec{x} + b \qquad ( EQ . 1 )$$

can be applied to the recorded sensor data to convert from AU/mV to mmHg. The scaling factor A modifies the gauge factor of the sensor, while factor b modifies the baseline. Using data from the FPS and arterial pressure sensor, the coefficients for four acutely implanted pigs were calculated. Coefficients were calculated to minimize the residuals for each recording.

The gauge factor varied widely across the different implanted sensors. The first two implants had a similar scaling factor and standard deviation; however, the values for implants 3 and 4 are extremely increased compared to the first two. Additionally, implant 4 has a wide standard deviation, ranging from 12737 to 23962. While the magnitude of gauge factor doesn't necessarily have to be consistent between sensors, the variation of gauge factor of a specific sensor must remain as stable as possible to enable a consistent representation of blood pressure. This essentially comes down to the stability of the CB-PDMS within the sensor. The b coefficients between all four samples were fairly consistent, ranging between 62-76 units.

### B. Finite Element Analysis

Given the results from in vivo characterization, changes were made to the mechanical design of the standard FPS to improve reliability and performance (the new sensor developed was the stretch sensor). Investigations into the materials and geometry of the FPS and the new stretch sensor, as well as their effects on the arterial wall were investigated using Finite Element Analysis (FEA).

### 1. Sensor Revisions

Further revisions were planned to improve the performance and longevity in vivo after noting early failures in large animal implantations. The first intended improvement was made to the materials and construction of the FPS. As the response of the sensors is based on the strain imparted from distension of the blood vessel, the bulk material of the sensor is made of a material with a low Young's Modulus. Young's Modulus, also known as the modulus of elasticity, describes the stiffness or rigidity of a material and is denoted by E. Young's Modulus is defined as the ratio of stress to strain in a material under linear, elastic deformation. Mathematically, it is expressed as:

$$E = \frac{σ}{ϵ} \qquad ( EQ . 2 )$$

where E is Young's Modulus, typically in units of GPa or MPa, σ is the applied force per unit area, in units of Pascals or N/m\hat{ }2, and ϵ is the resulting change in length relative to the original length (dimensionless). Young's Modulus is used to characterize how a material will deform under axial or tensile loading. A higher Young's Modulus indicates a stiffer material, meaning it will deform less under a given load. On the contrary, a lower Young's Modulus suggests a more flexible or compliant material. Decreasing the Young's Modulus not only increases the amount of strain the sensor experiences, increasing gauge factor, but it also allows it to impart less stress on the blood vessel.

Since the strain sensitive portion of the sensors is circumferential to the artery, a more accurate definition of stress in EQ. 2 would be based on the hoop stress equation:

$${σ}_{h} = \frac{P \cdot d}{2 \cdot t} \qquad ( EQ . 3 )$$

Where P is the internal pressure, d is the inner diameter, and t is the thickness of the material. Rearranging EQ 2, and combining with EQ. 3 we can obtain an equation that describes the deformation of the sensor under arterial pulsation:

$$σ = E ϵ \qquad ( EQ . 4 )$$

$$\frac{P \cdot d}{2 \cdot t} = E \frac{δ}{L} \qquad ( EQ . 5 )$$

$${δ}_{h} = \frac{P \cdot L \cdot d}{2 \cdot t \cdot E} \qquad ( EQ . 6 )$$

Where L is the length of the material is assumed to be the outer circumference of the artery (π·d).

$${δ}_{h} = \frac{π \cdot P \cdot {d}^{2}}{2 \cdot t \cdot E} \qquad ( EQ . 7 )$$

One thing to note about the sensors are that the strain sensitive portion of the sensors does not extend along the entire circumference of the sensor. If the material properties of the non-strain sensitive portion of the sensor were modified to focus strain energy from distention into the strain sensitive portion, then the sensor could become more strain sensitive. Given the parameters in EQ. 7, there are a few potential avenues for this to take place. Pressure experienced by the sensor, and the diameter of the sensor are not controllable, as those are based on the physiological environment the sensor is implanted in. This leaves two parameters, thickness, and Young's Modulus to modify. In the case of thickness, focusing the strain from one section of the sensor to another would require one to be much thicker than the other, which is not a realistic solution. For example, a 50× transmission of strain from the non-sensitive to sensitive region would require the non-sensitive region to be 50× thicker, or the sensitive region to be 50× thinner. This results in a sensor that is 35 mm thick on one side, assuming 0.7 mm nominal thickness, or 14 microns thick on the other side. The former is not realistically implantable, and the latter is not realistically manufacturable or robust.

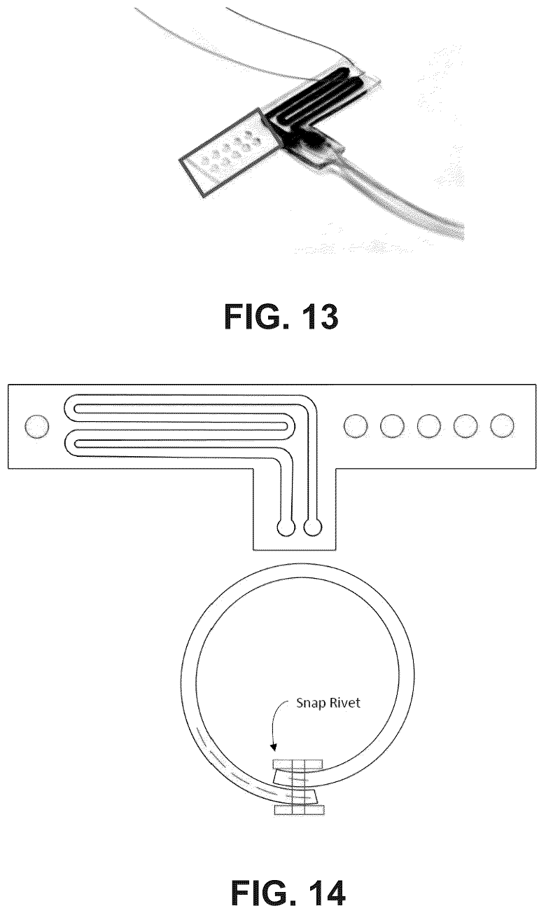

This leaves Young's Modulus as the last available parameter to modify. Young's Modulus of the PDMS can easily be controlled by introducing a stiffening element as a component of the non-sensitive portion of the sensor. This can be proven mathematically using equivalent spring constant formulas:

$${k}_{1} = {E}_{1} \frac{A}{π \cdot {d}_{0}} \qquad ( EQ . 8 )$$

$${k}_{2} = {k}_{21} + {k}_{22} = {E}_{1} \frac{A}{π \cdot {d}_{o}} + {E}_{2} \frac{A}{π \cdot {d}_{0}} \qquad ( EQ . 9 )$$

$${k}_{2} = ( {E}_{1} + {E}_{2} ) \frac{A}{π \cdot {d}_{0}} \qquad ( EQ . 10 )$$

$${δ}_{n} = \frac{F}{{k}_{n}} \qquad ( EQ . 11 )$$

$${δ}_{1} = \frac{F \cdot π \cdot {d}_{0}}{{E}_{1} \cdot A} \qquad ( EQ . 12 )$$

$${δ}_{2} = \frac{F \cdot π \cdot {d}_{0}}{( {E}_{1} + {E}_{2} ) \cdot A} \qquad ( EQ . 13 )$$

$$\frac{{δ}_{1}}{{δ}_{2}} = \frac{F \cdot π \cdot {d}_{0}}{{E}_{1} \cdot A} \cdot \frac{( {E}_{1} + {E}_{2} ) \cdot A}{F \cdot π \cdot {d}_{0}} \qquad ( EQ . 14 )$$

$$\frac{{δ}_{1}}{{δ}_{2}} = \frac{{E}_{1} + {E}_{2}}{{E}_{1}} \qquad ( EQ . 15 )$$

Where E₁ is the Young's Modulus for the PDMS, E₂ is the Young's Modulus for the stiffening element, do is the initial diameter of the sensors, A is the cross-sectional area, and F is the internal force within the sensors. This derivation shows that the portion of the sensor without the stiffening element will experience a higher proportion of strain relative to the non-stiffened portion at a ratio that relates the Young's Moduli of the two materials. Given this information, the stiffening element should be placed within the non-sensitive portion of the stretch sensor. This portion of the sensor is also where the attachment mechanism for the sensor is, so the stiffening element also has a secondary purpose of reinforcing the attachment area. FIG. 13 shows one of the placement regions for the stiffening element.

The second intended improvement was made to the closure mechanism of the FPS. In the current iteration of the FPS, sutures at the attachment site must be tightly attached to the silicone in order to ensure a robust closure. The force required from the sutures could potentially cause failure of the soft silicone body material. The proposed improvement involved transitioning to a snap rivet closure design for the stretch sensor. This approach was expected to reduce the amount of local stress on the silicone, offering a more efficient closure mechanism. The snap rivet design, shown in FIG. 14, aimed to provide a secure closure without the need for excessive force from sutures, and in doing so optimize the overall durability of the stretch sensor.

### 2. Methods

a. Sensor Geometry

To simulate the proposed revisions, geometric representations of the FPS and the new stretch sensor were designed in SolidWorks Student 2023 (Dassault Systèmes). Parameters for the geometry were referenced from mechanical drawings used for prior manufacturing. Parameters used are included below (Table 1).

TABLE 1
Parameters used to define the geometry of the sensor

| Parameter | Inner Diameter | Thickness | Suture Hole Diameter | Width |
|---|---|---|---|---|
| Value | 6.72 mm | 0.6 mm | 0.76 mm | 5.5 mm |

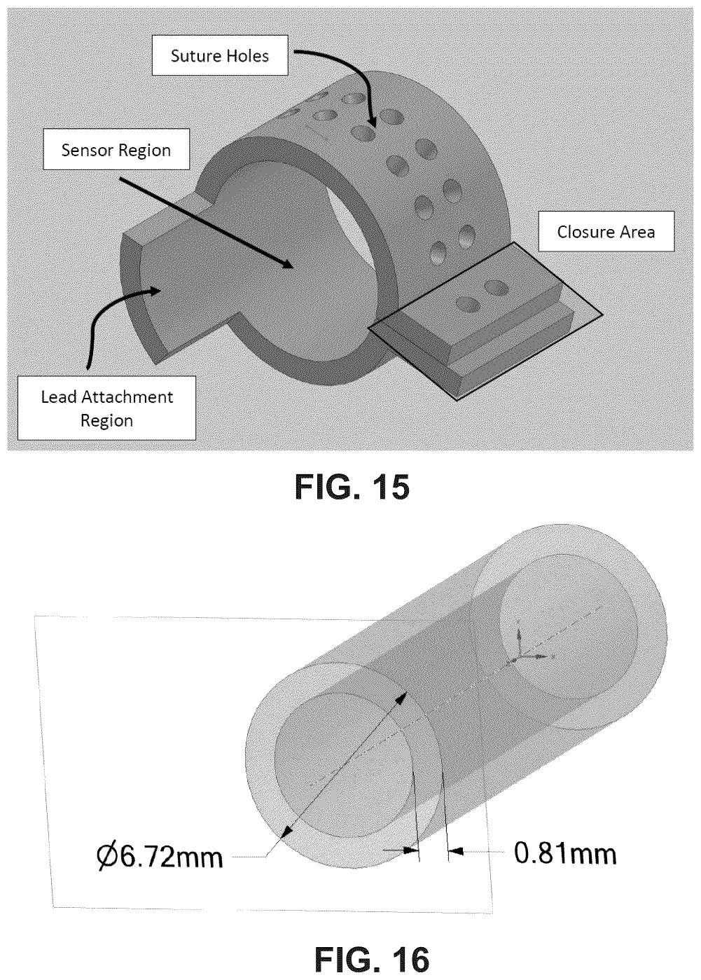

The FPS was modeled in a ‘duckbill’ closed state (see FIG. 15), this is one form that the FPS can take during implantation.

b. ANSYS FEA Setup

Ansys Mechanical was used as the FEA software for this analysis. ANSYS is a suite of finite element and computational fluid dynamics software developed by ANSYS Inc. It is widely used by engineers and designers to simulate the behavior of structures, components, and systems. Ansys Mechanical was used as the FEA software for this analysis. A simple model of a representative artery was designed in Ansys SpaceClaim according to the following parameters:

TABLE 2
Geometric Parameters of the simulated artery

| Parameter | Outer Diameter [mm] | Thickness [mm] |
|---|---|---|
| Value | 6.72 | 0.81 |

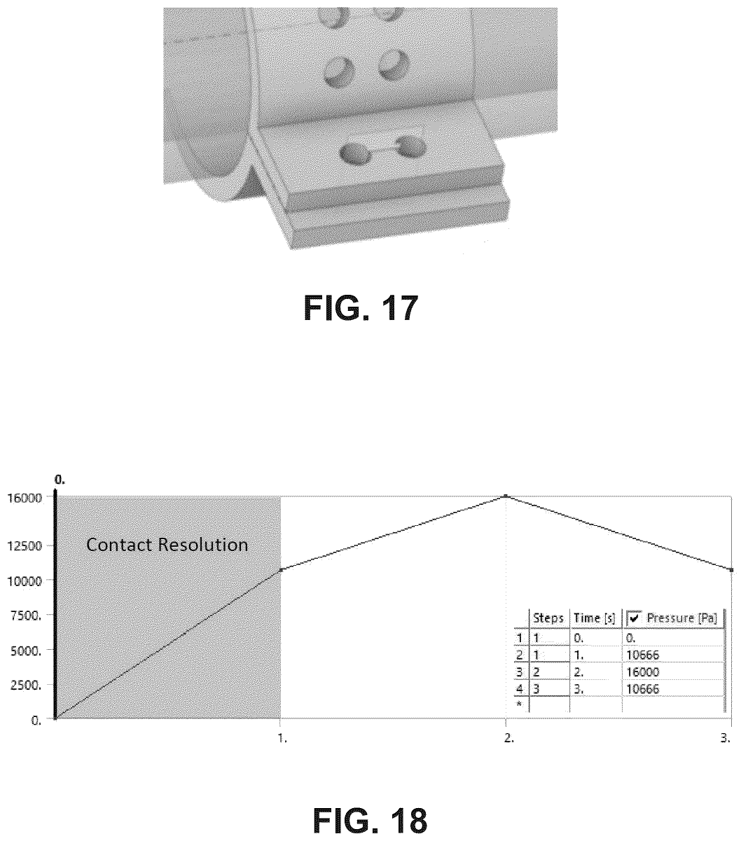

A simulated suture was also modeled as a beam profile around the interior of the suture holes at the attachment point (see FIGS. 16 and 17).

### 3. Material Properties

a. Arterial Physiology

The arterial walls are composed of three distinct layers, collectively known as the tunics, each contributing to the overall structure and function of blood vessels. The innermost layer, closest to the lumen, is the intima. Comprised of a single layer of endothelial cells supported by a thin layer of connective tissue, the intima facilitates a smooth surface for blood flow and helps regulate vascular tone and permeability. The middle layer, called the media, is predominantly composed of smooth muscle cells embedded in elastic fibers. This layer provides the artery with elasticity and contractility, allowing it to adapt to changes in blood pressure and regulate blood flow. The outermost layer, the adventitia, is composed of connective tissue containing collagen fibers that provide structural support and anchor the artery to surrounding tissues.

Arterial walls exhibit non-linear elastic (NLE) behavior due to the complex and dynamic nature of the materials composing them. The interaction between these components results in a non-linear stress-strain relationship, meaning that the arterial walls do not deform uniformly under varying levels of stress like a typical elastic material.

b. Moony Rivlin 5

To simulate a realistic arterial response to wall distension, actual material parameters were included in the system. Parameters were referenced from studies that simulated a similar large arterial model and are shown below:

TABLE 3

| | | | | | | Den- | |
|---|---|---|---|---|---|---|---|
| | | | | | | sity | |
| | a₁₀ | a₀₁ | a₂₀ | a₁₁ | a₀₂ | [g/ | Poisson's |
| | [MPa] | [MPa] | [MPa] | [MPa] | [MPa] | cm\hat{ }3] | Ratio |
| Value | 0.01890 | 0.00275 | 0.59042 | 0.85718 | 0 | 1.1 | 0.45 |

With Strain Energy density function:

$$W = {\sum }_{p , q = 0}^{n} {{a}_{pq} ( {I}_{1} - 3 )}^{p} {( {I}_{2} - 3 )}^{q} + {\sum }_{m = 0}^{M} \frac{1}{{D}_{m}} {( J - 1 )}^{2 m} \qquad ( EQ . 16 )$$

$$W = {a}_{10} ( {I}_{1} - 3 ) + {a}_{01} ( {I}_{2} - 3 ) + {a}_{11} ( {I}_{1} - 3 ) ( {I}_{2} - 3 ) + {{a}_{20} ( {I}_{1} - 3 )}^{2} + {{a}_{02} ( {I}_{2} - 3 )}^{2} + \frac{1}{{D}_{1}} {( J - 1 )}^{2} \qquad ( EQ . 17 )$$

Where I₁ and I₂ are two invariants of the left Cauchy-Green deformation tensor, and J is the determinant of the deformation gradient. This model did not simulate each of the layers of the arterial wall independently, instead opting for an aggregate representation of the entire arterial wall as one material. The FPS and the stretch sensor were simulated as a simple linear elastic material with varying Young's Modulus depending on scenario.

### C. Boundary and Loading Conditions

The following conditions were used as a baseline for simulation. The duckbill had a ‘bonded’ connection to simulate closure conditions, the interior surface of the FPS and the exterior surface of the artery had a ‘rough’ connection to simulate internal friction, and the suture had a ‘frictionless’ connection to allow the solver to calculate tension at the interface. The ends of the artery had a displacement constraint that prevents translation or rotation in space. The ends were defined as deformable, which still allowed them to expand in plane to prevent internal tension from affecting results. The suture also had a displacement constraint that prevents translation and rotation, but the behavior was defined as coupled, which allowed it to move in space with coincident nodes. This was useful as it allowed the duckbill closure to move in space without imparting force if necessary. Loading conditions were defined in 4 steps total, with one contact resolution step, and three force imparting steps. FIG. 18 shows the pressure profile used for distention of the artery in simulation. In FEA a contact resolution step is often necessary to accurately model and simulate interactions between separate components or bodies in a system. The contact resolution step is there to resolve any interferences and contacts in the Contact Resolution model and preload any deformations necessary. Properly resolving contact improves the numerical stability and convergence of the simulation. The pulsation waveform was simulated as a pressure force on the interior lumen of the artery. A simple linear interpolation of a pulsation between 80 mmHg and 120 mmHg represented the pulse wave.

### 4. Results

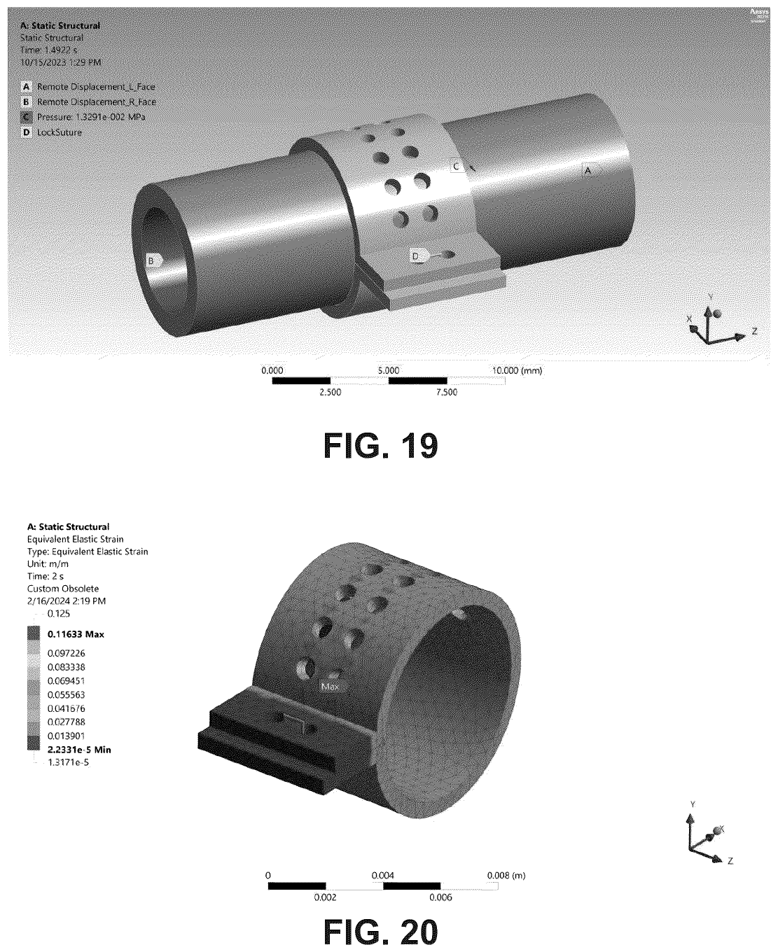

An initial model of the previous iteration of the FPS was tested in in vivo was prepared to serve as a baseline to compare improvements and modifications in the current iteration of the sensor. FIG. 19 shows an example geometric model of the previous version of the sensor. FIG. 20 illustrates the distribution of strain specifically at the point of maximum distension experienced by the sensor. The most notable occurrence in this model was the distribution of strain in the material. Strain energy in this version of the sensor was primarily distributed in the suture attachment holes of the sensor, with a peak strain of 0.116. Average strain in the strain sensing region of the FPS was approximately 0.0567, just under half of the peak strain in the sensor. Additionally, the strain was not evenly distributed across the sensor surface, with the edge of the sensor experiencing more strain than the center region.

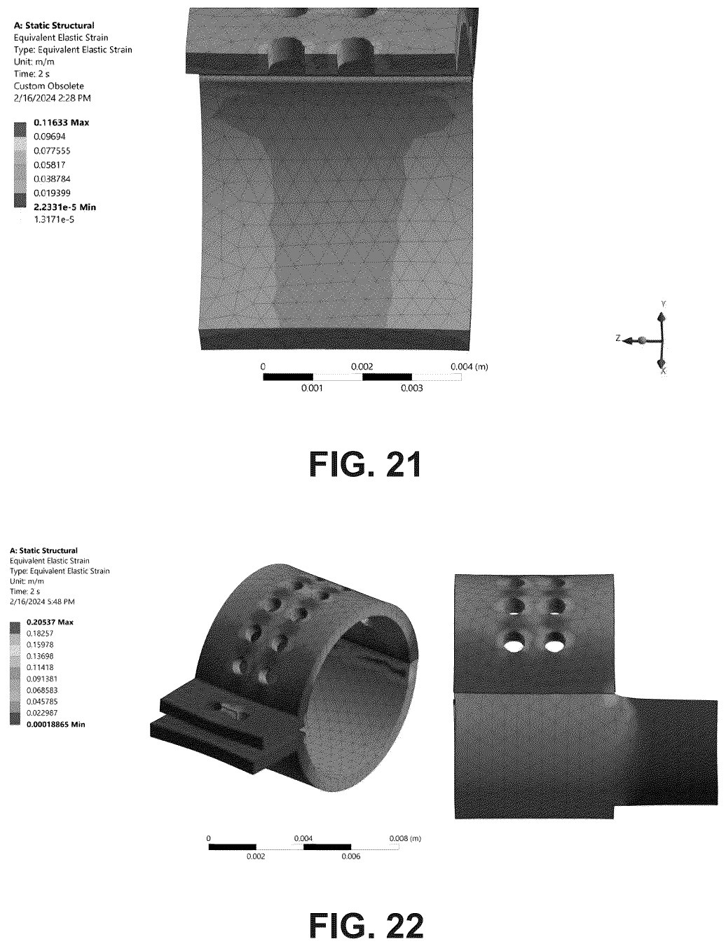

The observed strain distribution within the FPS holds some implications for the overall performance and functionality of the sensor. FIG. 21 shows an internal view of the FPS with notable strain distribution across the lumen of the sensor. The concentration of strain energy in the suture attachment holes suggests that these specific regions play a role in absorbing and responding to mechanical forces, potentially negatively influencing the overall durability and responsiveness of the sensor. This finding corroborated some of the findings during in vivo characterization and the concept of a stiffening element to improve the distribution of strain energy. The spatial variation in strain across the strain sensitive portion also prompted considerations regarding the uniformity and consistency of the sensor's sensitivity. The serpentine nature of the resistive pattern in the FPS partly accounted for this fact, but sensor improvement was still feasible based on FEA results.

a. Stiffening Element

The second analysis focused on the introduction of a stiffening element and a less stiff material for the strain sensitive portion of the new stretch sensor. The following properties defined the simulated materials:

TABLE 4
Mechanical properties used to represent an intended
softer silicon bulk material for the sensor.

| | Density | Young's Modulus | Poisson | UTS |
|---|---|---|---|---|
| Parameter | [kg/m\hat{ }3] | [kPa] | Ratio | [MPa] |
| Value | 965 | 43.3 | 4875 | 3.51 |

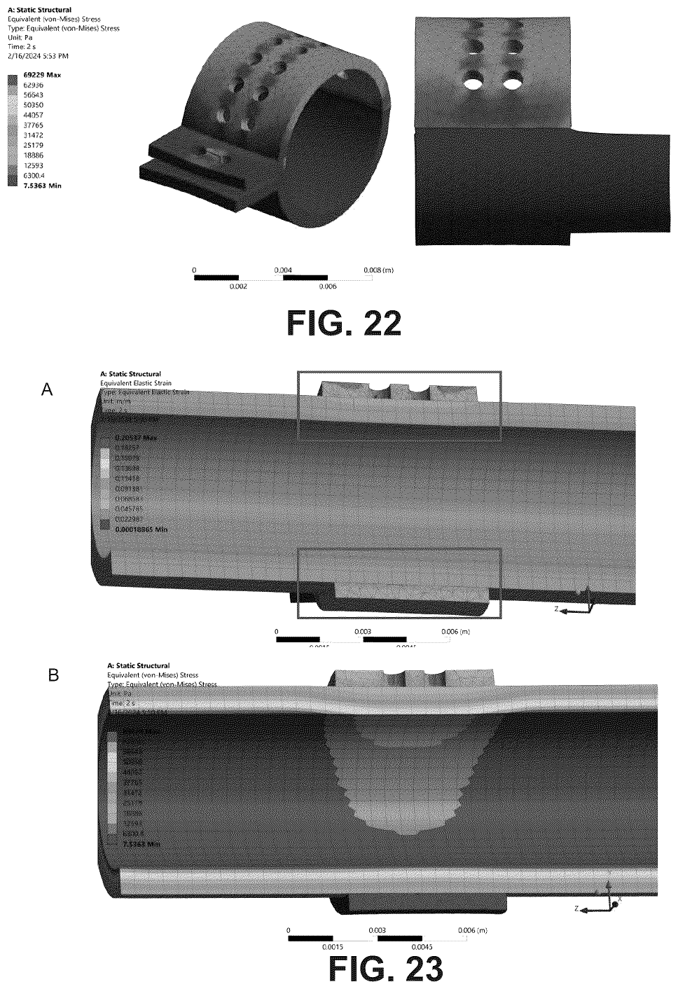

Due to the use of multiple bodies to enable split material analysis, some additional boundary conditions were considered. Similar to before, the interior surfaces of the partially FPS had a rough connection to the arterial wall, and each of the sections had a frictionless attachment to the suture. To investigate the effect of the solution on the closure mechanism, the bonded condition on the duck bill was disabled. Additionally, another bonded condition was added at the interface between the suture region and the sensing region to attach the two halves together. As expected, strain energy was primarily imparted into the strain sensitive portion of the sensor. Strain in the suture portion of the FPS averaged around 0.046, while strain in the sensor portion of the FPS averages about 0.091, which reversed the ratio in distribution of strain across the sensor from the baseline model (strain was about double in the strain sensitive portion). This iteration of the FPS also solved the issues with distribution of strain across the sensitive portion of the sensor, as there was no longer a strong spatial correlation to strain. The inclusion of the stiffening element introduced the side effect of a region of high strain at the interface between the two stiffnesses. This typically indicates an area where mechanical stress is concentrated; however, the low Young's Modulus of the material may have negated this stress concentration. FIG. 22 shows strain distribution of the sensor with the addition of the stiffening element. This configuration of the partially improved stretch sensor more efficiently utilized the forces in the strain sensitive portion, with all of the energy being used to cause distension. This is shown in the stress distribution of the partially improved stretch sensor, and the minimal amount of stress in the strain sensitive region. FIG. 22 shows stress distribution of the partially improved stretch sensor with the added stiffening element and FIG. 23 elements A and B show stress distribution of the artery during pulsation. The difference in stiffness also caused some asymmetrical loading on the artery; however, this loading appeared to be mostly negligible with respect to the range of forces during pulsation.

The closure mechanism, in the stress analysis in FIG. 22, demonstrated a noteworthy absence of stress concentration in comparison to the broader sensor structure. The force exerted on the closure mechanism was approximately 10 kPa, suggesting that the suture closure contributed minimally to the overall stress experienced by the sensor. This observation led to an interesting inference: the suture closure, in and of itself, does not emerge as a significant source of stress for the sensor; but rather, the presence of the suture holes to enable the closure to serve as local stress concentrators.

b. Snap Rivet

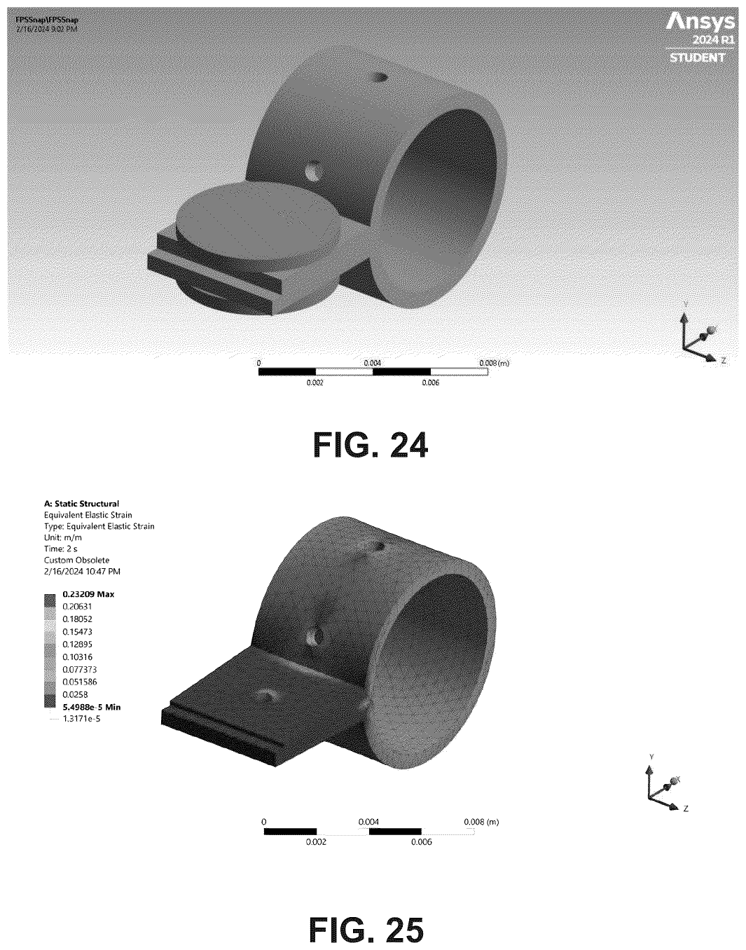

The snap rivet closure mechanism was designed to decrease the amount of local stress on the FPS by distributing the closure force over a larger area for the stretch sensor. By dispersing the force, the snap closure alleviated stress concentrations that could potentially compromise the structural integrity of the sensor. This simulation used the same material from the first model of the partially improved stretch sensor, with some changes to the geometry of the FPS in the closure region (see FIG. 24). The primary changes to the geometry were the replacement of the several pairs of suture holes with a series of singular holes, and the extension of the closure region to accommodate the larger size of the snap rivet closure. This meant that there was overall less adjustability in the nominal diameter of the partially improved stretch sensor, but the silicone body was expected to accommodate any stretch required in between holes. Boundary conditions were similar to those of the first model: a rough boundary condition between the sensor and the artery, and a frictionless condition between the sensor and the closure. Strain in the strain sensitive region of the sensor was averaged around 0.065. Strain was still focused around the closure mechanism attachment sites, but since the loss in strain energy was a function of the number of holes present in the sensor, the overall strain increased by 15% compared to the original design (0.065 vs. 0.057). The snap rivet design imparted about the same amount of strain at the closure site (see FIG. 21 & FIG. 25).

Linearity is a critical parameter, not just for the full-scale transfer characteristic but for the relationship between output signal and the signal to be measured. Non-linear responses may introduce systematic errors, jeopardizing the accuracy of measurements and potentially undermining the validity of the sensor. For the FPS and the stretch sensor, linearity is considered as the output response compared to the internal pressure of the artery. In a linear relationship, the change in output is directly proportional to the change in pressure, allowing for predictable results. To address non-linearity would require implementation of a calibration or mapping process to convert values between the non-linear response of the FPS and/or the stretch sensor and the linear response of blood pressure.

### C. Linearity

FPS linearity was estimated by sampling the simulation results at a specific node while changing the simulated pressure of the systolic phase in increments of 10 mmHg, from 0-160 mmHg. In the simulated response the low-pressure region of the stretch sensor (<60 mmHg) showed a region of slight nonlinearity; the functional range of the stretch sensor had a strong linear relationship between strain and blood pressure. This observation may be attributed to certain inherent characteristics of the blood vessel, or limitations of the sensor in low-pressure conditions. While this nonlinearity in the lower-pressure range was noteworthy, it fell within a non-physiologic region where the sensor is unlikely to be used. Additionally, during implantation it is highly likely that the sensor will be implanted with prestrain, translating the differential response of strain to be more similar to that of the functional range. While the inherent nonlinearity in the lower-pressure region is notable, the introduction of prestrain during implantation could potentially mitigate or compensate for this effect.

d. Encapsulation

The implantation of synthetic devices within the human body triggers a foreign body response, culminating in the formation of fibrous tissues surrounding the implanted object. This physiological reaction is characterized by the deposition of collagenous materials, resulting in the development of an encapsulation layer around the device. While this fibrous encapsulation is a natural defense mechanism, it can significantly impact the performance of sensors embedded near or within tissue. The encapsulation layer may introduce mechanical constraints and alter the local microenvironment, consequently influencing a sensor's output. Understanding these effects are pivotal considerations in the design and optimization of the new stretch sensor to ensure long-term functionality and minimize potential distortions caused by the foreign body response.

Fibrosis in the FEA simulation was modeled as a linear elastic material, with the following material and geometric properties:

TABLE 5
Material and geometric properties of the simulated fibrosis.

| | Youngs Modulus | Poisson's | Thickness |
|---|---|---|---|
| Parameter | [kPa] | Ratio | [mm] |
| Value | 60 | 0.3 | 0.4 |

Thickness is measured from the outer surface of the sensor.

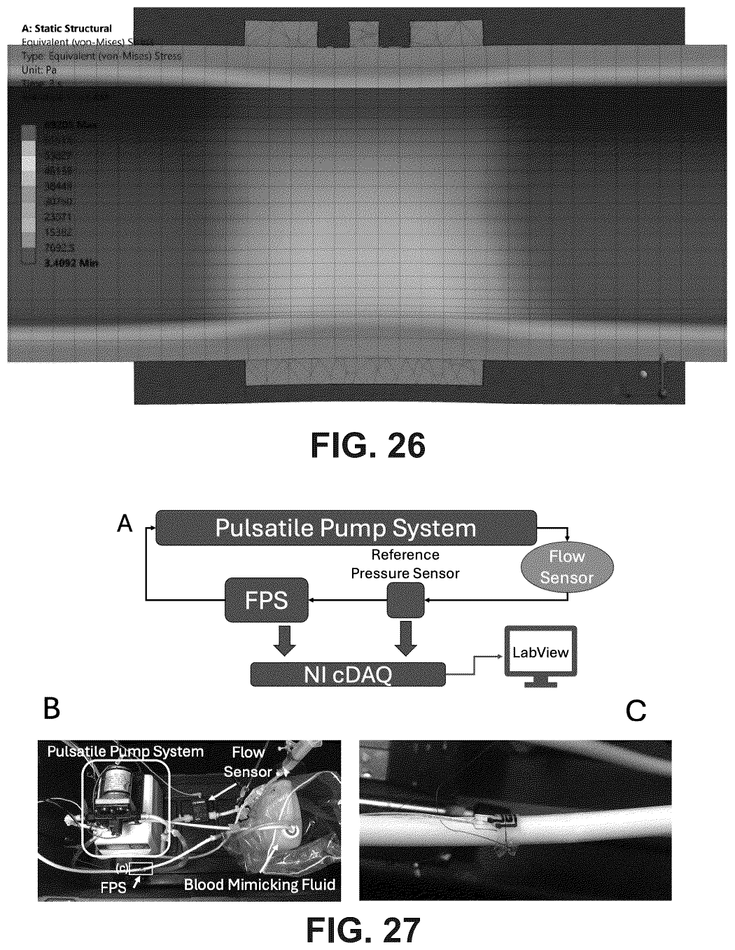

The geometries of the artery and sensor were subtracted from a simple cylinder to create the geometry of the encapsulation layer. The response of the artery to the encapsulation layer followed the typical response found in vivo. FIG. 26 shows stress response of the artery to encapsulation.

The fibrosis caused a local static increase in arterial pressure in the region of sensor implantation. This was consistent with the effects of vascular fibrosis, in which the increased stiffness of the arterial wall causes increased stress during systole. The increased stiffness also caused the strain ability of the region during pulsation to drop, with an average strain of 0.056, which was approximately a 2% difference from baseline (see FIG. 20). The primary outcome from this simulation confirmed the expected response of increased pressure in the artery due to stiffness, but the encapsulation appeared to have a minimal effect on the measurements made by the sensor. Interestingly, research has shown that the use of softer materials inhibits of the activation of TGF-ß1, which is the growth factor that leads to fibrotic encapsulation. This potentially indicates that the strain focused revision of the stress sensor may be more resilient to the effects of encapsulation in vivo, increasing longevity.

e. Autonomic Activation

Understanding and accounting for the impact of vasoconstriction due to autonomic activation is paramount in the context of the sensors. Vasoconstriction, a physiological response regulated by the autonomic nervous system, can significantly alter the diameter and compliance of blood vessels, directly influencing the strain experienced by arterial walls. Ignoring these dynamic changes may lead to inaccuracies in blood pressure measurements, as vasoconstriction can affect the baseline strain and alter the sensor's response. Consequently, incorporating considerations of autonomic activation-induced vasoconstriction into the stretch sensor design or signal processing pathway is crucial for ensuring the sensor's reliability across various physiological states. Autonomic activation was modeled. This caused an increase in stiffness/strain energy of the modeled material (EQ. 17). Extreme vasoconstriction was modeled by large changes in the a₁₀ parameter.

The response of the stretch sensor to autonomic activation was simulated. As expected, an increase in the stiffness of the artery resulted in a decrease in the amount of strain in the arterial wall. While this result would otherwise be inappropriately conflated with a change in blood pressure (i.e. when examining only the maximum strain), there are some ways to differentiate these effects. The primary method for differentiating vasoconstriction and blood pressure is to consider the location and magnitude of the dicrotic notch in the cardiac cycle. Other simulations have noted that increased elasticity and viscous effects lead to an earlier, and higher magnitude dicrotic notch due to changes in signal propagation and reflection within the artery.

c. Benchtop Characterization

Based on the results of FEA, the stretch sensor was designed. Benchtop characterization of the stretch sensor is crucial for understanding and quantifying performance. This process allows for the sensor to be tested under controlled conditions, assessing its sensitivity, accuracy, and reliability. It helps identify strengths and weaknesses and guides adjustments for optimal functionality. Benchtop characterization provides a measure for real-world applications, ensuring the sensor meets performance standards.

### 1. Setup

Benchtop characterization was performed via measurement of the stretch sensor while attached to a vascular phantom to assess long-term stability during continuous cycles of pulsating flow. A steady baseline and consistent response throughout constant use is essential for accurate and reliable blood pressure measurements. Additional mechanical characterization of the stretch sensor was also performed via uniaxial tensile testing until failure.

FIG. 27, element A shows a schematic of the testing system, FIG. 27, element B shows a photo of the vascular phantom, and FIG. 27, element C shows a photo of the system mounted on a silicone tube. In the vascular phantom, pressure and flow sensors verified that the pulsatile waveforms closely resembled human hemodynamics. Vessel phantoms comprised a 6 mm outer diameter silicone tube simulating an artery. The stretch sensor was wrapped around the phantom artery to sense pressure and flow (FIG. 27, element C). Data were sampled using National Instruments cDAQ-9401 and cDAQ-9218 modules, controlled by LabVIEW (2022 Q3). A lowpass median filter was applied as a smoothing filter to remove high frequency noise from the signal.

### 2. New Sensor Design

A new sensor design based on the results from FEA was developed to implement the new material and geometric findings. The bulk material of the sensor was selected to be Ecoflex™ 00-10 due to its softness, low elastic modulus, biocompatibility, and ease of use. The manufacturing process for the new sensor primarily remained the same as introduced below, with a few key changes.

Fabrication of the stretch sensor (shown in FIG. 28) is as follows:
A. Silicone is cast over a positive mold, this creates a channel in the pattern the resistive material must take.
B. The casting is removed from the mold. A laser cut mylar sheet stencil is placed on top of the silicone.
C. CB-PDMS composite material is placed into the channel. PtOIr are embedded into the CB-PDMS to establish electrical contact.
D. A single part room-temperature-vulcanizing (RTV) silicone was added at the interface to the electrical contact leads to increase resistance to the leads pulling out.
E. A final layer of silicone is case over all layers to seal the sensor.
F. The belt loop closure mechanism is punched out of the silicone for securing the stretch sensor to an artery.

Metal shims were used to define layer thickness, and the final shape of the stretch sensor was cut out at the end using stencils. To implement the stiffening element explored in the FEA analysis, a nylon spandex mesh (90% nylon, 10% elastane) was embedded into the closure region of the stretch sensor at or between steps D and E. The mesh can easily be interchanged for other materials, such as surgical mesh to optimize the stiffness of the stretch sensor per application. Stainless steel leads were also used for connection to the CB-PDMS.

FIG. 29 shows a photo of the mylar stencil used to define the resistive pattern (element A), and the new revision of the sensor with updated bulk material and stiffening element (element B). A new composition for the resistive layer was also employed to increase the sensitivity to distension, integrating carbon nanotubes with the existing carbon black particles to adjust conductivity. Fabrication directions for the new composition are listed here.

### Fabrication of CB-PDMS Mix

1. Grind carbon black granules with mortar and pestle. Add desired amount of carbon black granules and carbon nanotubes to an empty beaker.
2. Add 10 g of Vertrel to beaker.
3. Sonicate mixture for 20 min (500 W, 20 KHz, 20% duty cycle)
4. Add 4.3 g of Ecoflex 00-10 Part A and 4.3 g of Ecoflex 00-10 Part B to beaker.
5. Add 10 g of Vertrel to beaker and mix by hand for 2-3 min.
6. Sonicate mixture for 20 min (500 W, 20 KHz, 20% duty cycle)
7. Pour mixture into aluminum tray and magnetically stir until mixture becomes too viscous to stir (˜20 min)
8. Transfer tray to vacuum desiccator and degas for 20-30 min (until about 3% of Vertrel remains, visually check for any excess solvent)

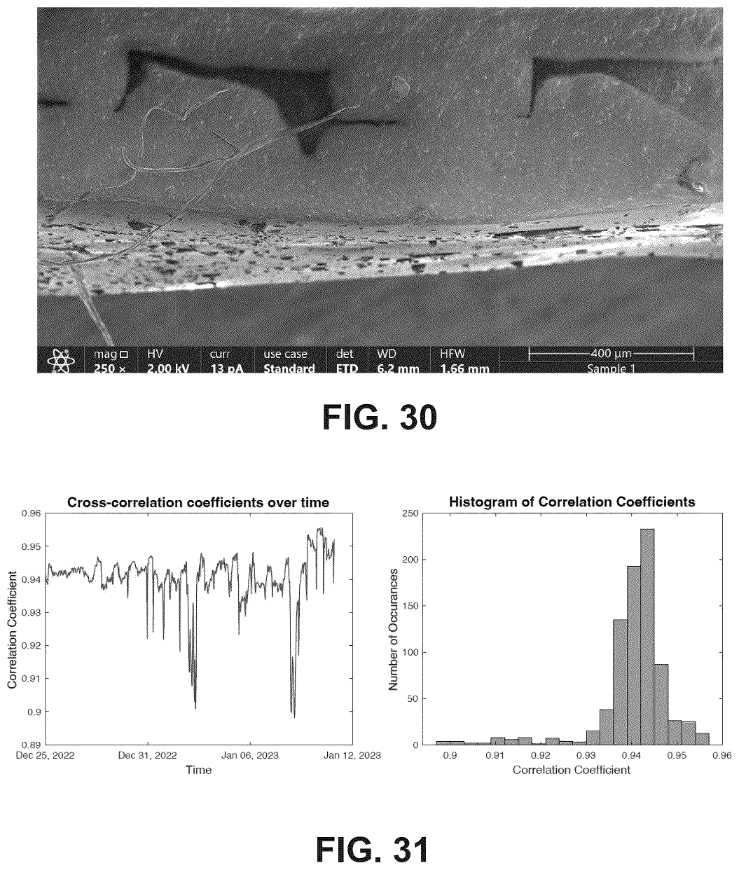

The new composition adjusted the ratio of carbon black nanoparticles to carbon nanotubes, while keeping the total amount of carbon in the resistive layer the same. The composition as tested was 80% CB nanoparticles (0.96 g) and 20% carbon nanotubes (0.24 g). A cross-sectional view of the new sensor is shown in FIG. 30. FIG. 30 shows a SEM imaging of the cross section of the stretch sensor. The low Young's Modulus of the new material leads to compression and uneven distribution of the internal traces when the topcoat of silicone is applied.

### 3. Phantom Results

a. Longevity

Two parameters, offset and sensitivity drift, were the primary focus during this testing phase. Offset drift refers to any gradual deviation from the initial baseline measurement over time, while sensitivity drift pertains to changes in the sensor's responsiveness to arterial distention. Values were compared to a reference blood pressure sensor (Deltran ©, Utah Medical Products). Pressure testing for the original sensor design was performed continuously over 17 days, at physiological blood pressure conditions (120/80 mmHg) for >1.5 million simulated cardiac cycles. The test ended when the reference blood pressure sensor failed while the stretch sensor continued to function.

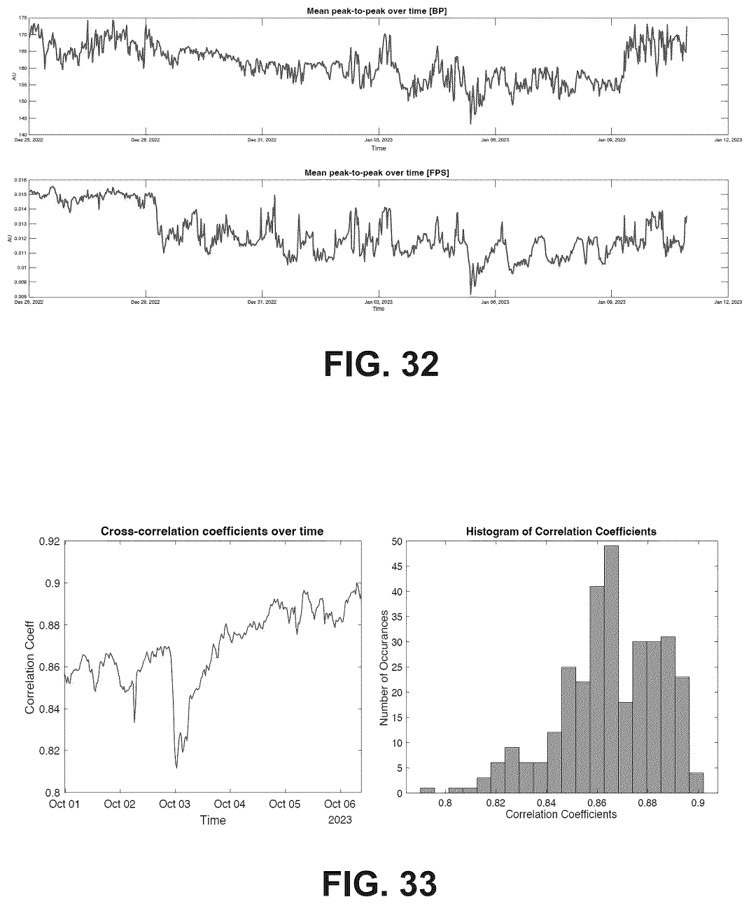

Data were segmented into 30-minute increments for analysis. Signal amplitude and mean value for both the reference pressure sensor and stretch sensor were calculated. Cross-correlation coefficients for each of the parameters were also calculated. FIG. 31 shows the cross-correlation coefficients for the recorded values of the pressure sensor and the stretch sensor over the duration of several weeks. Correlation remains consistently strong through the sampling duration. Throughout the duration of recording, the stretch sensor demonstrated enhanced stability, as evidenced by a deviation in the mean of approximately 0.1% of the full range, equating to roughly 1.4 mmHg of pressure. This low deviation/offset drift indicated a high level of consistency and reliability in the stretch sensor's measurements of blood pressure. To determine sensitivity drift, the average peak-to-peak was calculated for each segment. In the context of determining sensitivity drift, this metric is particularly insightful, as any fluctuations or deviations in the peak-to-peak values can signify alterations in the sensor's responsiveness or gauge factor. Changes in the reference pressure sensor were predominantly reflected in the output of the stretch sensor, with major features of the trend corresponding between the two (see FIG. 32). The strong correlation coefficients between the reference pressure sensor and the stretch sensor suggest that the data conveyed by the stretch sensor can be confidently compared to a clinical gold standard. FIG. 33 shows the cross-correlation of longevity testing of the new sensor design. The sensor has a peak coefficient of 0.9, which remains a strong correlation.

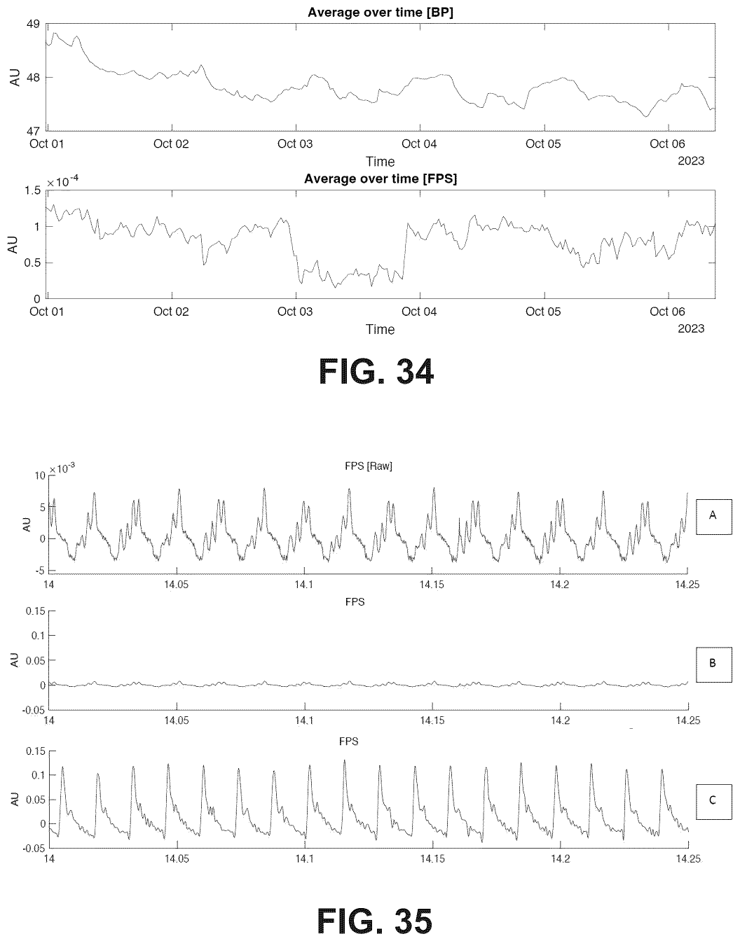

Longevity testing for the revised sensor design was performed continuously over 6 days, also at physiological blood pressure conditions (120/80 mmHg). As shown in FIG. 34, an artifact appeared between October 3 and October 4 that caused the average value and cross correlation of the stretch sensor to drop in that period. Otherwise, it appeared that the distribution of correlation coefficients was centered about 0.86, though it had an upwards trend starting on October 4. The reference sensor used for comparison exhibited what is most likely a subtle temperature influence, apparent in the low-frequency oscillations occurring once each day. The stretch sensor demonstrated no inherent temperature dependence; however, this influence could have resulted in a decrease of correlation.

Taking a representative sample of data from each revision, the response range/gauge factor of the stretch sensor increased with the new construction. The original sensor (FIG. 35, elements A and B) had a response range of approximately +5 mV, while the new sensor (FIG. 35, element C), had a response range of +75 mV, which is about a 15× increase in the gauge factor. In the context of the stretch sensor, a 15× increase in the gauge factor can have several implications and significance. Firstly, it implies improved resolution and accuracy in measuring pressure-related strain, which is crucial for obtaining more precise data on arterial dynamics. This heightened sensitivity could enable the detection of subtle changes in pressure or strain, making the sensor more effective in capturing variations associated with cardiovascular conditions. It could improve the sensor's ability to detect early signs of vascular abnormalities, such as subtle changes in blood flow patterns or the onset of conditions like stenosis.

b. Flow Calculation

The correlation between flow and pressure within the arterial system is a fundamental aspect of cardiovascular physiology. In the context of blood circulation, flow and pressure are linked and influence each other. Flow, typically represented as the volume of blood passing through a specific point or cross-sectional area per unit time, is directly proportional to the pressure difference across the venous return. According to the principles of fluid dynamics, blood flows from regions of higher pressure to lower pressure. Thus, variations in arterial pressure create a driving force for blood flow. Understanding the correlation between flow and pressure is vital for assessing cardiovascular health. Abnormalities in this relationship, such as increased resistance or impaired vessel elasticity, can signify underlying cardiovascular conditions.

However, the stretch sensor is primarily sensitive to changes in pressure within the vessel rather than direct measurements of blood flow. In situations such as arterial stenosis, where there is a narrowing of the artery, the resistance to blood flow increases. This, in turn, leads to an elevation in pressure upstream from the stenotic region. The strain sensor, detecting changes in the arterial wall deformation, captures variations in pressure resulting from altered flow dynamics. It is important to note that while changes in pressure can be indicative of changes in flow, the sensor directly measures strain and pressure, not the volumetric flow rate. Therefore, stretch sensor measurements likely require calibration to each implanted scenario, depending on the anatomy and underlying atherosclerosis of the host vessel.

While the stretch sensor primarily captures changes in pressure, it is still possible to infer and estimate flow dynamics from recorded values. The relationship between pressure and flow in the circulatory system is governed by fundamental principles of fluid dynamics. These estimations may not provide direct volumetric flow measurements but can offer insight into the relative changes in flow patterns. Additionally, by understanding the interplay between pressure and flow within the context of the specific vascular environment, meaningful information about the hemodynamic status of the artery can still be used to infer potential alterations in blood flow associated with conditions like stenosis or other vascular abnormalities.

Flow was measured using an Omega Electromagnetic Flow Meter (OMEGA Engineering. The flowmeter works by outputting a delta-sigma encoded signal at a rate that correlates with the volume of fluid moving through the unit. The LabView program records the number of impulses and further processing via a moving average filter is used to obtain arbitrary units of flow. First, a calibration curve between the units of the arbitrary units of flow and volumetric flow rate (mL/min) was calculated. The calibration curve was generated by running a measured volume of fluid through the phantom over a given period of time, (e.g. 400 mL measured over 1 minute). The corresponding flow value measured by LabView was also recorded over this period, and results are plotted below. The recorded flow value had a linear response to volume up until approximately 825 mL/min. At this point, it is most likely that internal resistances to flow in the phantom were limiting the output of the system. This flow rate is beyond the flow rate of most peripheral arteries in humans, however.

With this information, flow values measured during pulsation of the stretch sensor were correlated to the output of the stretch sensor, providing an estimate of volumetric flow through the artery based on the output of the sensor. By simultaneously recording data from both sensors during phantom pulsation, a correlation between the strain sensor's pressure-related measurements and the directly measured volumetric flow can be established. Leveraging this correlation (especially within the linear response range of the flow meter) enabled the development of a conversion between the units recorded by the strain sensor and the calibrated volumetric flow values so flow can be estimated.

### 4. Tensile Test Results

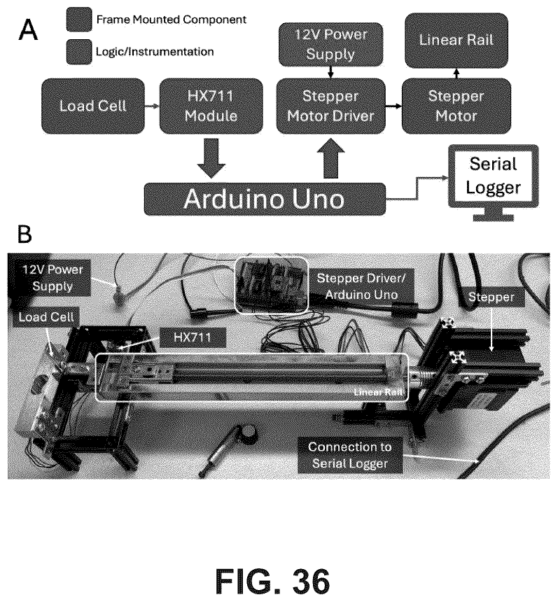

Stress strain curves were generated to compare material response between new and old sensor materials. Uniaxial tension testing was performed to generate this data. A test apparatus (shown in FIG. 36) was constructed using a calibrated load cell (HX711), load cell amplifier, and Arduino Uno for instrumentation. A Velmex precision linear slide was used to adjust the position of the stage. A stepper motor was used to control the position of the linear slide and was driven by a A4988 stepper motor driver module with microstepping capabilities driven by a 12V power supply. Microstepping enabled high precision control of the test stage, down to a linear resolution of 0.00625 in (˜0.16 mm). Hardware components of the tension tester were mounted a frame built from aluminum extrusions.

a. Sensor Materials

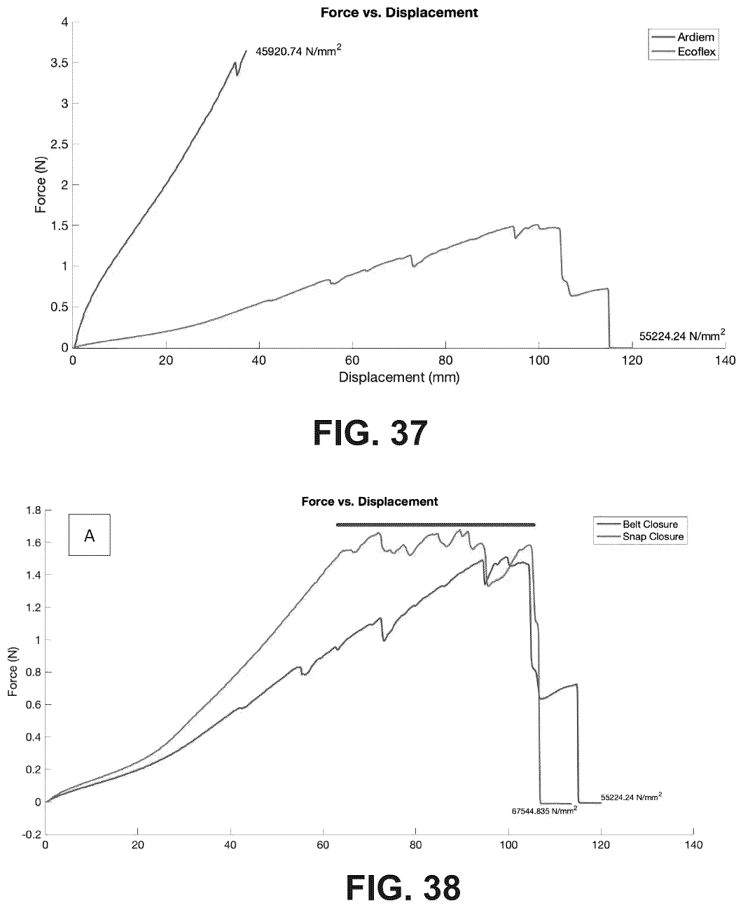

Tensile pulls were conducted to measure the response of the suture belt loop closure, snap rivet closure, and connection of the leads to the body of the sensor. All tests were conducted at a linear rate of 1.52 mm/s. The first series of test considered the belt loop configuration of closures. FIG. 37 shows the stress strain diagram between the original sensor (top line) and the new design (lower line). The original sensor is plotted in blue, and the new design is plotted in orange. The integral of the stress strain curve is notated at the end of each trace and corresponds to the toughness of the configuration. Toughness is the ability of the material to absorb energy under applied forces. Between the two designs, the new materials used in constructing the sensor greatly increased the range of strain before failure. This means that more of the energy from distention is transduced to the sensor in a way that decreases reaction forces on the artery. While the stretch sensor does not experience acute distention in the range of 20 mm, the increased range and toughness of the material is indicative of increased mechanical longevity in vivo. The heightened toughness suggests that the sensor can withstand greater levels of strain, deformation, or external forces without compromising its structural integrity. The findings suggest that the new stretch sensor design has the potential to offer prolonged and reliable performance. The increased material toughness and expanded strain tolerance position the sensor to maintain structural integrity and functionality over an extended period, even in challenging physiological environments.

b. Sensor Closure

The next test compared the closure mechanisms when used with the new material composition. Stress-strain testing evaluates how well these closures can withstand and distribute mechanical forces, identifying potential weaknesses. Both sensors failed at approximately 105 mm of deformation, with tearing of the material in the snap closure version of the sensor, indicated by a plateau between 60 mm and failure.

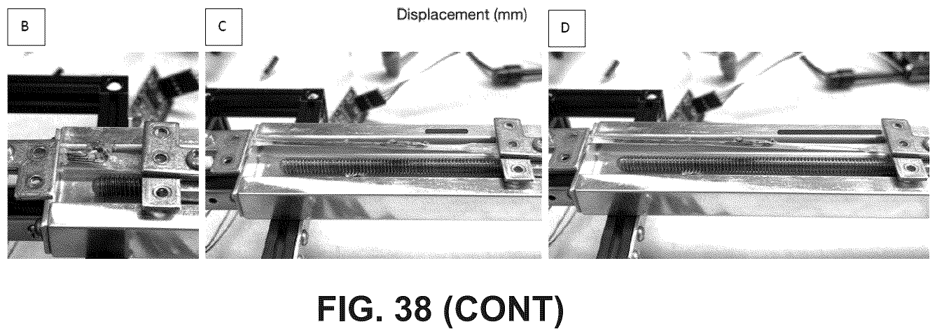

While the overall topography of the graph of FIG. 38 is generally the same, the snap closure modification resulted in an increase in toughness over 22%. These results were consistent with results from FEA, which predicted strain energy to be lost into the holes used for the suture closure. Importantly, the mesh used to divert strain energy into the sensitive portion of the stretch sensor appears work as intended, as that portion of the sensor does not elongate much in the tension tester over the course of deformation (FIG. 38, elements B-D). Additionally, the FEA also predicted the correct failure modes for the closure mechanisms, with the suture holes resulting in failure in the closure region, and the snap closure resulting in failure at the interface between the two stiffnesses of the sensor.

### C. Lead Connection

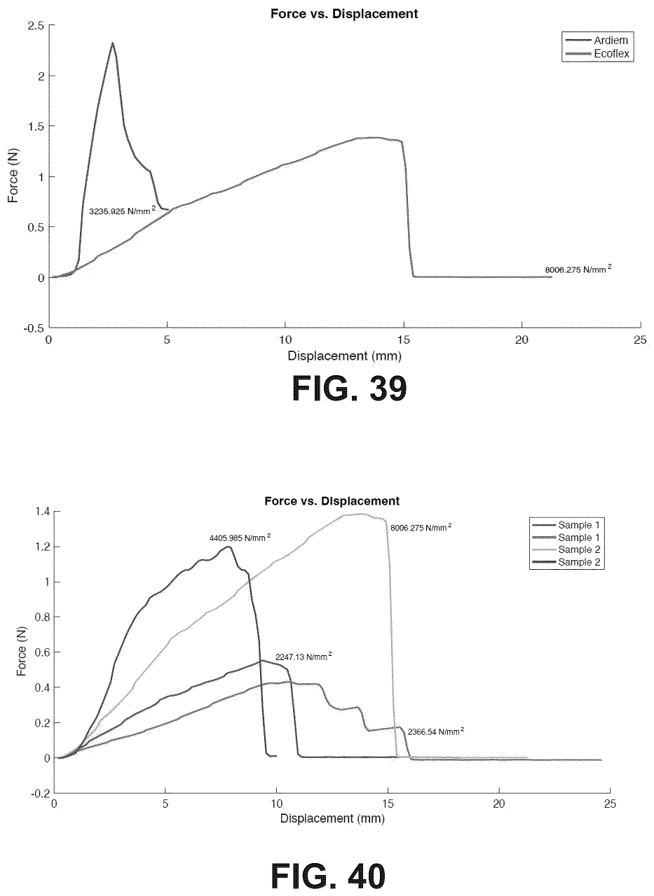

The final tension test compared the response of the lead interface to external forces (see FIGS. 39 and 40). Some of the interference and connection issues found during in vivo testing could be attributed to the quality of the connection of the leads to the CB-PDMS, and their degeneration over time. The comparison of the original and new sensor designs revealed substantial improvements in the resilience and toughness of the lead interface. The original sensor, characterized by a displacement of approximately 2.7 mm before pulling out also exhibits a relatively stiff interface, as indicated by the steep initial curve leading to failure. In contrast, the new sensor design demonstrated an enhancement in toughness, registering an almost 2.5× increase compared to the original design. This heightened toughness suggests a significant improvement in the interface's ability to absorb energy before failure. Additionally, the softer bulk material of the new sensor design allowed for a substantially greater displacement of approximately 15 mm before the lead pulled out from the body. The combination of increased toughness and higher displacement capacity in the new design indicated a more resilient lead interface. This enhanced resilience is indicative of the interface's improved ability to endure external forces, absorb energy, and maintain structural integrity in the face of mechanical challenges.

To confirm the effect of RTV silicone on the resilience of the interface, tests were performed to compare the response of the leads with and without RTV silicone applied. RTV silicone was not applied at the interface of Sample 1, whereas Sample 2 received the application of RTV silicone. The application notably increased the toughness of the interface by a minimum factor of 2. The added silicone did not necessarily increase the range of displacement before failure as evidenced by the purple trace. There appeared to be an inverse correlation between the modulus of the interface and the range of displacement before failure.

From the above description, those skilled in the art will perceive improvements, changes and modifications. Such improvements, changes and modifications are within the skill of one in the art and are intended to be covered by the appended claims.

What is claimed is:

1. A sensor comprising:
a flexible substrate configured to wrap around at least a portion of an artery and comprising a sensing portion in an intermediate portion of the flexible substrate and a non-sensing portion split between opposite ends of the flexible substrate;
the sensing portion comprises a piezoresistive material comprising conductive particles suspended in a polymer and configured to detect changes in a diameter of the artery;
at least one lead configured to interface with the sensing portion to transmit the detected changes in the diameter of the artery to a controller;
the non-sensing portion comprises at least one opening positioned longitudinally on each of the opposite ends of the flexible substrate and a stiffening mesh around each of the at least one opening on each of the opposite ends of the flexible substrate; and
one or more closure mechanisms configured to connect the opposite ends of the flexible substrate via the at least one opening on each of the opposite ends of the flexible substrate.

2. The sensor of claim 1, wherein the one or more closure mechanisms comprises a snap-rivet closure configured to secure the opposite ends of the flexible substrate through the at least one of the plurality of openings.

3. The sensor of claim 2, wherein the snap-rivet closure prevents tearing of the non-sensing portion due to stresses on the sensor.

4. The sensor of claim 1, wherein the stiffening mesh is embedded within:
a layer attached over and/or under at least one of the opposite ends of the flexible substrate, and/or
the non-sensing portion of the flexible substrate.

5. The sensor of claim 1, wherein the stiffening mesh comprises 90% nylon and 10% elastane.

6. The sensor of claim 1, wherein the flexible substrate wraps around at least the portion of the artery by the sensing portion in the intermediate portion of the flexible substrate at least partially encircling the at least the portion of the artery and the non-sensing portion on the opposite ends of the flexible substrate closing around the at least the portion of the artery.

7. The sensor of claim 6, wherein the non-sensing portion on one of the opposite ends of the flexible substrate is configured to overlap the non-sensing portion on another of the opposite ends of the flexible substrate such that openings on each of the opposite ends of the flexible substrate overlap and the one or more closure mechanisms connect through openings to secure the flexible substrate around at least the portion of the artery.

8. The sensor of claim 1, wherein the piezoresistive material comprises 80% carbon black nanoparticles and 20% carbon nanotubes.

9. The sensor of claim 1, wherein the flexible substrate comprises a soft, biocompatible material that provides an increased compliance compared to deformation of a wall of the artery.

10. The sensor of claim 9, wherein the increased compliance inhibits the activation of growth factors that lead to fibrotic activation and allows for the sensor to be sensitive to the deformation of the wall of the artery.

11. The sensor of claim 9, wherein the material comprises a platinum-catalyzed silicone material.

12. The sensor of claim 1, further comprising a room-temperature-vulcanizing (RTV) silicone interface between the at least one lead and the sensing portion to secure the at least one lead to the flexible substrate.

13. The sensor of claim 1, wherein an output of the sensor is linear compared to an internal pressure of the artery and is directly proportional to a change in the internal pressure.

14. A system comprising:
a sensor comprising:
a flexible substrate configured to wrap around at least a portion of an artery and comprising a sensing portion in an intermediate portion of the flexible substrate and a non-sensing portion split between opposite ends of the flexible substrate,
the sensing portion comprises a piezoresistive material comprising conductive particles suspended in a polymer and configured to detect changes in a diameter of the artery,
the non-sensing portion comprises at least one opening positioned longitudinally on each of the opposite ends of the flexible substrate and a stiffening mesh around each of the at least one opening on each of the opposite ends of the flexible substrate, and
one or more closure mechanisms configured to connect the opposite ends of the flexible substrate via the at least one opening on each of the opposite ends of the flexible substrate; and
a controller in communication with the sensor, wherein the controller comprises at least a processor configured to execute instructions to determine measurements related to arterial pressure based on recordings from the sensor and output the measurements related to the arterial pressure.

15. The system of claim 14, further comprising a display in communication with the controller and configured to visualize the measurements related to the arterial pressure.

16. The system of claim 14, wherein the instructions to output the measurements comprise:
receive data related to strain of the sensing portion of the sensor;
determine an internal pressure of the artery based on the data; and
output a blood pressure-related reading based on the internal pressure of the artery.

17. The system of claim 16, wherein the data related to strain of the sensing portion of the sensor comprises an electrical resistance measurement.

18. The system of claim 17, wherein the controller is further configured to execute instructions to determine one or more cardiovascular variability parameters based on at least one blood pressure reading.

19. The system of claim 17, wherein the controller is further configurated to execute instructions to determine and output an estimation of volumetric blood flow rate based on a plurality of the blood pressure readings over a time.

20. The system of claim 14, wherein the controller is in wireless communication with the sensor.
